SEQUENCE LISTING (8 sequences)

SEQ ID NO: 1 - RNA, 65 nt, Homo sapiens
  misc_feature: Human pre-miR-155
cuguuaaugcuaaucgugauagggguuuuugccuccaacugacuccuacauauuagcauuaacag

SEQ ID NO: 2 - RNA, 23 nt, Homo sapiens
  misc_feature: Human Mature miR-155
uuaaugcuaaucgugauaggggu

SEQ ID NO: 3 - RNA, 65 nt, Mouse
  misc_feature: Mouse pre-miR-155
cuguuaaugcuaauugugauagggguuuuggccucugacugacuccuaccuguuagcauuaacag

SEQ ID NO: 4 - RNA, 23 nt, Mouse
  misc_feature: Mouse Mature miR-155
uuaaugcuaauugugauaggggu

SEQ ID NO: 5 - DNA, 20 bp, Artificial Sequence
  Synthetic primer
gtaacccgttgaaccccatt

SEQ ID NO: 6 - DNA, 20 bp, Artificial Sequence
  Synthetic primer
ccatccaatcggtagtagcg

SEQ ID NO: 7 - DNA, 20 bp, Artificial Sequence
  Synthetic primer
caccaccatcaaggactcaa

SEQ ID NO: 8 - DNA, 20 bp, Artificial Sequence
  Synthetic primer
aggcaacctgaccactctcc

(19) United States
(12) Patent Application Publication
(10) Pub. No.: US 2011/0293653 A1
(43) Pub. Date: Dec. 1, 2011

(54) ANTAGONISTS OF MIR-155 FOR THE TREATMENT OF INFLAMMATORY LIVER DISEASE

(75) Inventors: Gyongyi SZABO, Southborough, MA (US); Shashi BALA, Shrewsbury, MA (US); Miguel MARCOS-MARTIN, Salamanca (ES)

(73) Assignee: UNIVERSITY OF MASSACHUSETTS, Boston, MA (US)

(21) Appl. No.: 13/046,534

(22) Filed: Mar. 11, 2011

Related U.S. Application Data
(60) Provisional application No. 61/313,000, filed on Mar. 11, 2010.

(51) Int. Cl. A61K 31/713; A61P 1/16; C12N 5/0786; A61K 36/06

(52) U.S. Cl. 424/195.16; 514/44 A; 435/375

(57) ABSTRACT

Provided herein are methods of treating or preventing an inflammatory liver disease in a subject, such as alcoholic liver disease (ALD), by administering to said subject an miR-155 antagonist.

### RELATED APPLICATIONS

This application claims priority to U.S. Provisional Patent Application No. 61/313,000, filed Mar. 11, 2010. The entire contents of the foregoing application is incorporated herein by reference.

### GOVERNMENT SUPPORT

This invention was made with government support under grant Nos. AA011576 and AA008577 awarded by the National Institute on Alcohol Abuse and Alcoholism. The United States Government has certain rights in the invention.

### BACKGROUND OF THE INVENTION

Inflammatory liver disease (hepatitis) is a significant medical problem. Chronic inflammation can lead to extensive liver damage and scarring of the liver (i.e., cirrhosis), liver failure or hepatocellular carcinoma. As a group, inflammatory liver diseases are characterized by the presence of inflammatory cells in the liver tissue and are often associated with overproduction of TNFα and other inflammatory cytokines. Chronic hepatitis can be caused by a host of factors including viral infection, environmental toxins (e.g., drugs), autoimmunity, or genetic mutation.

Alcoholic hepatitis (also known as alcoholic liver disease (ALD)) is a common medical consequence of chronic alcohol abuse. The pathogenesis of acute and chronic alcohol consumption is multi-factorial with diverse consequences in different cell types. Alcohol-induced injury occurs at multiple levels ranging from innate immune cells (e.g., hepatic macrophages or Kupffer cells) to the liver parenchymal cells (hepatocytes). The currently accepted model of alcoholic liver injury elucidates that LPS promotes hepatic injury via induction of Kupffer cell activation resulting in the production of TNFα and other inflammatory mediators. Kupffer cells respond to stimulation by gut-derived endotoxins (e.g., LPS) and apoptotic dead cells in the tissue, resulting in increased inflammatory responses (see Mandrekar & Szabo, J. of Hepatology, 1258-1266, 2009).

Metabolic disorders can also cause different forms of hepatitis. Non-alcoholic fatty liver disease (NAFLD) is fatty inflammation of the liver which is not due to excessive alcohol use. The incidence of NAFLD is increasing dramatically, particularly in the Western world, and can lead to an increase in the prevalence of nonalcoholic steatohepatitis (NASH), the most extreme form of NAFLD, as well as associated complications such as cirrhosis and hepatocellular carcinoma (HCC). NASH can also be associated with obesity, diabetes and insulin resistance.

Many inflammatory liver diseases (e.g., ALD, NAFLD or NASH) lack a specific treatment. Moreover, previous clinical trials employing anti-TNFα antibodies for the treatment of these disorders have been unsuccessful due to the significant risk of increased infection as a result of TNF blockage. Accordingly, alternative therapies for the treatment of inflammatory liver diseases are urgently needed.

### SUMMARY OF THE INVENTION

Provided herein are methods of treating or preventing an inflammatory liver disease, such as alcoholic liver disease (ALD), in a subject. In particular, the invention is based, at least in part, on the surprising discovery that miR-155 antagonists attenuate the symptoms and pathology of inflammatory liver disease without completely blocking TNFα function. Accordingly, by causing only partial blockage of TNFα production, the methods of the invention provide a means for preventing inflammatory overactivation while preserving host immunity in the liver.

In certain aspects, the invention provides methods of treating or preventing an inflammatory liver disorder, comprising identifying a subject having, or suspected of having an inflammatory disorder, and administering to said subject a miR-155 antagonist.

Based at least in part on the above observation, the invention features, in a first aspect, a method for treating or preventing an inflammatory liver disease, which includes identifying a subject having, or at risk of having, an inflammatory liver disease, and administering to the subject a miR-155 antagonist in an amount effective to decrease expression of miR-155 in the subject, wherein the miR-155 antagonist partially suppresses TNFα expression, thereby treating or preventing the disease.

In another aspect, the invention features a method for decreasing the stability of TNFα mRNA in a target cell, which includes administering a miR155 antagonist to the cell in an amount effective to decrease expression of miR-155 in the cell, thereby decreasing the stability of TNFα mRNA in the cell.

In one embodiment of the above aspects, the inflammatory liver disorder is selected from alcoholic liver disorder (ALD), non-alcoholic fatty liver disease (NAFLD) and non-alcoholic steatohepatitis (NASH). In a preferred embodiment the above aspects, the inflammatory liver disorder is ALD.

In another embodiment of the above aspects, the miR-155 antagonist is an anti-miR155 antisense oligonucleotide or an RNAi agent. In a preferred embodiment of the above aspects, the antisense oligonucleotide is an antagomir, an LNA oligonucleotide, or an 2′O-methyl antisense RNA oligonucleotide. In another preferred embodiment of the above aspects, the RNAi agent is a siRNA or a shRNA.

In an additional embodiment of the above aspects, the miR-155 antagonist is complementary to a sequence at least 80% identical to human mature miRNA-155. In another embodiment of the above aspects, the miR-155 antagonist is complementary to a sequence at least 80% identical to pre-microRNA-155. In yet another embodiment of the above aspects, the miR-155 antagonist is perfectly complementary to a human microRNA-155 seed sequence.

In a further embodiment of the above aspects, the miR-155 is administered in a pharmaceutical composition comprising a yeast cell wall particle (YCWP).

In another embodiment of the above aspects, the target cell is a Kupffer cell or a macrophage or a hepatocyte.

In yet another aspect, the invention pertains to the methods of detecting liver damage (e.g., from acute or chronic liver disease) by detecting increased levels of miR-122 or miR-155 in samples from subjects (e.g., in serum or plasma). In one embodiment, these markers can be detected at an early stage of liver damage, e.g., prior to onset of cancer.

Other features and advantages of the invention will be apparent from the following detailed description, and from the claims.

### BRIEF DESCRIPTION OF THE DRAWINGS

FIG. 1. Graphically depicts the enhanced miR155 expression in RAW macrophages after LPS and/or ethanol treatment. As depicted in FIG. 1(A) RAW 264.7 macrophages were stimulated with 50 mM ethanol for the indicated time points. As depicted in FIG. 1(B) RAW 264.7 macrophages were stimulated with 50 mM ethanol for 6 hours, with LPS for 6 hours or with LPS for 6 hours after 48 hours of ethanol pretreatment. Expression of miR-125b, miR-146a and miR155 was assayed by qPCR and data were normalized to sno202 control. The fold increase in the expression of these miRNAs versus non-stimulated cells is shown. Data represent the mean value (s.e.m. as error bars) of at least three independent experiments. Statistically significant differences are shown (* indicates p<0.05 versus unstimulated cells).

FIG. 2. Graphically depicts the increase in TNF-α production in RAW macrophages after LPS and/or ethanol treatment and correlates with miR155 expression. As depicted in FIG. 2(A) RAW 264.7 macrophages were stimulated with 50 mM ethanol for the indicated time points and TNF-α levels were measured in supernatants by ELISA. As depicted in FIGS. 2(B and C) RAW 264.7 macrophages were stimulated with 50 mM ethanol for 6 hours, with LPS for 6 hours or with LPS for 6 hours after 48 hours of ethanol pretreatment. TNF-α levels were measured in supernatants by ELISA and TNF-α mRNA was quantified using specific primers in real-time PCR. Data represent the mean value (s.e.m. as error bars) of at least three independent experiments. Statistically significant differences are shown. As depicted in FIG. 2(D) the correlation between miR155 expression and TNF-α production in RAW 264.7 macrophages under different conditions (50 mM ethanol for 6, 24 and 48 hours and 100 ng/mL LPS for 6 hours with or without ethanol pretreatment) is shown (R2=0.94, p<0.01). Expression of miR155 was assayed by qPCR and data were normalized to sno202 control. TNF-α levels were measured in supernatants by ELISA after collection of the media in the same samples. Each dot represents the average of at least three independent experiments.

FIG. 3. Illustrates that chronic ethanol feeding induced liver steatohepatitis in mice as well as increased ALT, alcohol and endotoxin serum levels. Mice (15 per group) received the Lieber-DeCarli diet for 4 weeks as described in Materials and Methods. Blood was collected after every week of feeding and serum was separated and analyzed for (A) alcohol, (B) ALT, and (C) endotoxin levels. Mean values with s.e.m. as error bars are shown for n=10. (* indicates p<0.05 when compared with pair-fed mice). FIG. 3(D) depicts representative sections of formalin-fixed, paraffin-embedded livers stained with hematoxylin and eosin of each group.

FIG. 4. Graphically depicts that chronic ethanol feeding increased TNF-α production in mice Kupffer cells. Kupffer cells isolated from pair-fed and ethanol-fed mice were pooled (n=5 per group) and cultured for 8 hours, followed by stimulation with 0 or 100 ng/ml LPS for 6 hours. As depicted in FIG. 4(A) TNF-α levels were measured in supernatants by ELISA after collection of the media. Data represent the mean value (s.e.m. as error bars). As depicted in FIG. 4(B) total RNA was isolated and analyzed for mRNA levels of TNF-α using specific primers in real-time PCR. Values of relative TNF-α mRNA expression normalized for housekeeping gene 18s are shown as mean (s.e.m. as error bars).

FIG. 5. Graphically depicts chronic ethanol feeding enhanced miR155 expression in mice Kupffer cells. Kupffer cells isolated from pair-fed and ethanol-fed mice were pooled (n=5 per group), cultured for 14 hours and harvested. Total mRNA was extracted and expression of miR-125b, miR-146a and miR155 was assayed by qPCR. Data were normalized to sno202 control and the fold increase in the expression of these miRNAs in Kupffer cells from ethanol-fed mice versus Kupffer cells from pair-fed mice is shown. Data represent the mean value (s.e.m. as error bars). Statistically significant differences are shown (*=p<0.05 versus Kupffer cells from pair-fed mice).

FIG. 6. Graphically depicts the induction of miR-155 in livers and hepatocytes of alcohol-fed mice. C57BL/6 mice (8-10/group) received the Lieber-DeCarli diet with 5% (vol/vol) of ethanol or isocaloric liquid control diet for four weeks. After four weeks of feeding, total livers or hepatocytes were isolated. Total RNA was isolated and used to quantify miR-155 expression by real time PCR. The values were normalized to Sno-202 (endogenous control) or miR-16 (serum samples) and are shown as the fold-increase over the pair-fed control group. Data represent mean values S.D. (* p<0.05 compared to pair-fed mice).

FIG. 7. Graphically depicts that miR155 increases TNF-α secretion by means of affecting TNF-α mRNA stability. As depicted in FIG. 7(A) RAW 264.7 macrophages were transfected with anti-miR-155 or anti-miR control. As depicted in FIG. 7(B) RAW 264.7 macrophages were transfected with pre-miR-155 or pre-miR precursor negative control. The cells were exposed to 50 mM ethanol for 48 hours and further stimulated with 100 ng/mL LPS for 6 h. Culture medium was collected and supernatants analyzed for TNF-α production by ELISA. Mean values of TNF-α (s.e.m. as error bars) from three independent experiments are shown. FIG. 7(C) graphically depicts RAW 264.7 macrophages transfected with anti-miR-155 or anti-miR-control, exposed or not to 50 mM ethanol for 48 hours, stimulated with 100 ng/mL LPS for 1 h and further cultured in the presence of 5 μg/mL actinomycin D. Total RNA was isolated at the times shown and TNF-α mRNA was quantified using specific primers in real-time PCR. Data were normalized for housekeeping gene 18s and are shown as percentage of remaining TNF-α at different time points. Shown is data (mean±s.e.m.) from an experiment out of three with similar results.

FIG. 8. Graphically depicts that treatment with NF-κB inhibitor MG-132 prevented miR155 increase in response to LPS and/or ethanol stimulation. RAW cells were exposed or not to ethanol (50 mM) and MG-132 (0.25 μM) for 48 hours and were further stimulated with LPS (100 ng/mL) for 2 hours. Expression of miR155 was assayed by qPCR and data were normalized to sno202 control. The fold increase in the expression of miR155 versus nonstimulated cells is shown. Data represent the mean value (s.e.m. as error bars) of at least three independent experiments. Statistically significant differences are shown.

FIG. 9. Graphically depicts the induction of miR-155 in serum of alcohol-fed mice. C57BL/6 mice (8-10/group) received the Lieber-DeCarli diet with 5% (vol/vol) of ethanol or isocaloric liquid control diet for four weeks. After four weeks of feeding, blood was collected and serum was separated at the time of scarification. Total RNA was isolated and used to quantify miR-155 expression by real time PCR. The values were normalized to Sno-202 (endogenous control) or miR-16 (serum samples) and are shown as the fold-increase over the pair-fed control group. Data represent mean values±S.D. (* p<0.05 compared to pair-fed mice).

FIG. 10. Graphically depicts increased serum miR-122 levels after alcohol feeding. C57BL/6 mice (5-8/group) received the Lieber-DeCarli diet with 5% (vol/vol) of ethanol or isocaloric liquid control diet for 2 or 4 weeks. After 2 or 4 weeks of feeding, blood was collected and serum was separated and stored at −80° C. Total RNA was isolated from the serum and used to quantify miR-122 expression by Taq-Man real time PCR (left). The values were normalized to miR-16 (endogenous control) and are shown as the fold-increase over the pair-fed control group. Alanine aminotransferase (ALT) was measured from the serum of corresponding animals (middle). Correlation between serum miR-122 and ALT was performed by Pearson method (right). Data represent mean values±S.D. (* p<0.05 compared to pair-fed mice.)

FIG. 11. Graphically depicts the induction of serum miR-122 in CCL-4-induced liver injury model. C57BL/6 mice (3-4/group) received either corn oil or CCl4 (IP; 0.6 ml/kg of body weight) for the indicated times, blood was collected and serum was separated at the time of scarification and stored at −80° C. Total RNA was isolated from the serum and used to quantify miR-122 as described above (left). Alanine aminotransferase (ALT) was measured from the serum of corresponding animals (middle). Correlation between serum miR-122 and ALT was performed by Spearman method (right). Data represent mean values±S.D. (* p<0.05 compared to untreated mice.)

FIG. 12. Graphically depicts that NADPH oxidase-deficient (p47^{phox} KO) mice showed no increase in serum miR-122 and ALT after 4 weeks of alcohol feeding. C57BL/6 mice (6-8/group) received the Lieber-DeCarli diet as mentioned above. Total RNA was isolated from the serum and used to quantify miR-122 as described above (left). Alanine aminotransferase (ALT) was measured from the serum of corresponding animals (right). Data represent mean values±S.D.

FIG. 13. Graphically depicts the reduction of miR-155 in livers of TLR4 KO after alcohol feeding. C57BL/6 mice (8-10/group) received the diet as mentioned above (FIGS. 6 and 9) and total RNA was analyzed for miR-155 expression as described earlier (FIGS. 6 and 9). Data represent mean values±S.D.

FIG. 14. Graphically depicts the increased expression of miR-155 in livers of MCD-fed mice. C57BL/6 mice (6-8/group) received the MCS or MCD diet for the time as indicated and total RNA was isolated from the livers and analyzed for miR-155 expression as described above (FIGS. 6 and 9). Data represent mean values±S.E.

FIG. 15. Graphically depicts the induction of miR-155 in hepatocytes, liver mononuclear cells (MNCs) and Kupffer cells of MCD-fed mice. C57BL/6 mice (6-8/group) received the MCS or MCD diet for 5 weeks, and heaptocytes (left), MNCs (middle) and Kupffer cells (right) were isolated. The next day, cells were treated or not with 100 ng/ml LPS for 6 h and total RNA was isolated and analyzed for miR-155 expression as described above (FIGS. 6 and 9). Data represent mean values±S.E.

FIG. 16. Graphically depicts the correlation (Pearson test) between miR-155 and TNF alpha in livers of MCD-fed mice C57BL/6 mice (6-8/group) received the MCS or MCD diet for 5 weeks. Total RNA was isolated from the livers and analyzed for miR-155 and TNF alpha expression as described above. Data represent mean values±S.E.

### DETAILED DESCRIPTION OF THE INVENTION

The invention is based, at least in part, on the discovery that miR155 is a key regulator of TNFα in inflammatory liver disease. In particular, the data presented herein demonstrate that changes in TNF-α production mirror miR155 levels in macrophages after ethanol and/or LPS stimulation. Contrary to prior reports which suggested that miR-155 is a negative regulator of alcohol-induced liver disease (see e.g., Yeligar et al., J. Immunol., 2009, 183: 5232-5243), the data provided herein support the surprising conclusion that alcohol alone can induce miR-155 over-expression and that miR-155 overexpression exerts a positive regulation on the release of tumor necrosis factor (TNF)-α by stabilizing TNFα mRNA and enhancing its translation. Consequently, the present invention implicates miR155 as an important therapeutic target for the treatment of ALD and other inflammatory liver diseases.

In another aspect, the invention is also based on the discovery that inhibition of miR155 can prevent the increase in liver TNF-α levels that occur as result of environmental stimuli (e.g., ethanol consumption and/or LPS). In particular, the invention provides the surprising finding that miR-155 antagonists (e.g., nucleic acid-based miR-155 antagonists) can partially block TNFα inflammation of the liver while preserving its essential immune functions in the liver. Accordingly, the miRNA antagonists of the invention can be used for the manufacture of a improved medicament for the treatment of an inflammatory liver disease.

In yet another aspect, the invention pertains to the methods of detecting liver damage (e.g., from acute or chronic liver disease) by detecting increased levels of miR-122 or miR-155 in samples from subjects (e.g., in serum or plasma). In one embodiment, these markers can be detected at an early stage of liver damage, e.g., prior to onset of cancer.

### I. DEFINITIONS

So that the invention may be more readily understood, certain terms are first defined.

As used herein, expression is “upregulated” or “increased” when the amount of RNA, or of a polypeptide encoded by the RNA, present in a cell or biological sample is greater than the amount of RNA, or of a polypeptide encoded by the RNA, present in a control cell or biological sample. Likewise, expression of an RNA is “downregulated” or “decreased” when the amount of RNA, or of a polypeptide encoded by the RNA, present in a cell or biological sample is less than the amount of RNA, or of a polypeptide encoded by the RNA, present in a control cell or biological sample.

The term “miRNA antagonist,” as used herein, refers to an agent that reduces or inhibits the expression, stability, or activity of a miRNA (e.g., miR155). A miRNA antagonist may function, for example, by blocking the activity of a miRNA (e.g., blocking the ability of a miRNA to function as a translational repressor and/or activator of one or more miRNA targets), or by mediating miRNA degradation. Exemplary miRNA antagonists include nucleic acids, for example, antisense locked nucleic acid molecules (LNAs), antagomirs, or 2′O-methyl antisense RNAs targeting a miRNA.

In certain embodiments, a miRNA antagonist of the invention may be nucleic acid, including, for example a RNA molecule, a DNA molecule, a hybrid DNA/RNA molecule, or an analog thereof (e.g., an RNA analog). The term “RNA” or “RNA molecule” or “ribonucleic acid molecule” refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double-stranded, i.e., dsRNA and dsDNA, respectively).

The term “RNA analog” refers to an polynucleotide (e.g., a chemically synthesized polynucleotide) having at least one nucleotide analog as compared to a corresponding unaltered or unmodified RNA but retaining the same or similar nature or function as the corresponding unaltered or unmodified RNA. The oligonucleotides may be linked with linkages (or internucleoside linkage groups) which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Preferred RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to unmodified RNA that it has the ability to mediate the same desired function (e.g., inhibition of miRNA function).

The term “nucleoside” refers to a nucleic acid molecule having a purine or pyrimidine base covalently linked to a ribose or deoxyribose sugar. Exemplary nucleosides include adenosine, guanosine, cytidine, uridine and thymidine. The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms.

The term “nucleotide analog” or “altered nucleotide” or “modified nucleotide” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function. Examples of positions of the nucleotide which may be derivitized include the 5 position, e.g., 5-(2-amino)propyl uridine, 5-bromo uridine, 5-propyne uridine, 5-propenyl uridine, etc.; the 6 position, e.g., 6-(2-amino)propyl uridine; the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs such as those described in Herdewijn, Antisense Nucleic Acid Drug Dev., 2000 Aug. 10(4):297-310. Nucleotide analogs may also comprise modifications to the sugar portion of the nucleotides. For example the 2′OH-group may be replaced by a group selected from H, OR, R, F, Cl, Br, I, SH, SR, NH₂, NHR, NR₂, COOR, or OR, wherein R is substituted or unsubstituted C₁-C₆ alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions which allow the nucleotide to perform its intended function such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) preferably decrease the rate of hydrolysis of, for example, polynucleotides comprising said analogs in vivo or in vitro.

The nitrogeneous base of a nucleotide analog may also be modified. The term “nitrogenous base” refers to purines and pyrimidines, such as the DNA nucleobases A, C, T and G, the RNA nucleobases A, C, U and G, as well as non-DNA/RNA nucleobases, such as 5-methylcytosine (MeC), isocytosine, pseudoisocytosine, 5-bromouracil, 5-propynyluracil, 5-propyny-6-fluoroluracil, 5-methylthiazoleuracil, 6-aminopurine, 2-aminopurine, inosine, 2,6-diaminopurine, 7-propyne-7-deazaadenine, 7-propyne-7-deazaguanine and 2-chloro-6-aminopurine, in particular MeC. It will be understood that the actual selection of the non-DNA/RNA nucleobase will depend on the corresponding (or matching) nucleotide present in the target RNA sequence. For example, in case the corresponding nucleotide is G it will normally be necessary to select a non-DNA/RNA nucleobase which is capable of establishing hydrogen bonds to G. In this specific case, where the corresponding nucleotide is G, a typical example of a preferred non-DNA/RNA nucleobase is MeC.

In certain embodiments, the internucleoside linkage group of a nucleic acid may be modified. The term “internucleoside linkage group” refers to a group capable of covalently coupling together two nucleobases in a polynucleotide. Preferred examples include phosphate, phosphodiester groups and phosphorothioate groups. The internucleoside linkage may be selected form the group consisting of: -0-P(O)2—O—, -0-P(O7S)2-O—, -0-P(S)₂—O—, —S—P(O)2—O—, —S—P(O,S)—O—, —S—P(S)2—O—, -0-P(O)₂—S—, —O—P(O,S)—S—, —S—P(O)2-S—, —O—PO(RH)—O—, O—PO(OCH3)—O—, —O—PO(NRH)—O—, -0-PO(OCH2CH2S—R)—O—, —O—PO(BH3)-O—, —O—PO(NHRH)—O—, —O—P(O)2—NRH—, —NRH—P(O)2—O—, —NRH—CO—O—, —NRH—CO—NRH—, and/or the internucleoside linkage may be selected form the group consisting of: -0-C0-0-, —O—CO—NRH—, —NRH—CO—CH2˜, —O—CH2-CO—NRH—, —O—CH2-CH2-NRH—, —CO—NRH—CH2—, —CH2-NRH—CO—, —0-CH2-CH2-S—, —S—CH2-CH2-O—, —S—CH2-CH2-S—, —CH2-SO2—CH2—, —CH2—CO—NRH—, —O—CH2—CH2—NRH—CO—, —CH2—NCH3-O—CH2—, where RH is selected from hydrogen and C1-4-alkyl.

As used herein, the term “isolated” (e.g., “isolated mRNA”, “isolated miRNA” or “isolated RNAi agent”) refers to molecules which are substantially free of other cellular material, or culture medium when produced by recombinant techniques, or substantially free of chemical precursors or other chemicals when chemically synthesized.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

Various methodologies of the instant invention include steps that involves comparing a value, level, feature, characteristic, property, etc. to a “suitable control”, referred to interchangeably herein as an “appropriate control”. A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing a given methodology, as described herein. For example, a transcription rate, mRNA level, translation rate, protein level, biological activity, cellular characteristic or property, genotype, phenotype, etc. can be determined prior to introducing a compound (e.g., a miR155 antagonist; a compound that increases, upregulates, enhances or mimics expression of a gene or gene product that is targeted by miR155) of the invention into a cell or organism. In certain embodiments, a suitable control is a value, level, feature, characteristic, property, etc. determined in a cell or organism, e.g., a cell or organism afflicted with alcoholic liver disease, in the absence of a miR155 antagonist. In methodologies that involve initiating alcoholic liver disease in a cell or organism, the properties of a “suitable control” or an “appropriate control” can also be determined in cells or organisms that are healthy or do not have alcoholic liver disease. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

As used herein, the terms “inflammatory liver disorder”, “inflammatory liver disease”, or “hepatitis” refer to abnormalities associated with inflammation of the liver. In exemplary embodiments, the inflammatory liver disorder is associated with the overexpression of inflammatory cytokines, e.g., Tumor Necrosis Factor (TNFα).

The term “subject” includes humans, and non-human animals amenable to therapy, e.g., preferably mammals and animals susceptible to cancer, such as non-human primates, transgenic animals, dogs, cats, horses, and cows. The term “subject” also includes patients, more preferably patients having, or suspected of having, an inflammatory liver disorder, e.g., alcoholic liver disease (ALD), non-alcoholic fatty liver disease (NAFLD) or non-alcoholic steatohepatitis (NASH). The term “subject” may also refer to a cell or a tissue, preferably a cell or a diseased tissue.

The term “treatment” refers to a process, manner or regimen which allows for medicinal or surgical care for an illness or injury in a subject. In certain embodiments, the treatment comprises diminishing or alleviating at least one symptom directly or indirectly associated with or caused by an inflammatory liver disease or disorder, including, for example, alcoholic liver disease. For example, treatment can be diminishment of one or several symptoms of an inflammatory liver disease or disorder or complete eradication of an inflammatory liver disease or disorder, including, for example, alcoholic liver disease.

The term “treatment regimen” refers to a regulated course of treatment intended to preserve or restore health, or to attain some result, e.g., inhibit or suppress an inflammatory liver disease or disorder, including, for example, alcoholic liver disease. In one embodiment the treatment regimen may include administering a miRNA antagonist, preferably, a miR155 antagonist. In a further embodiment, the treatment regimen may include administering an anti-inflammatory agent and a miR155 antagonist to a subject.

As used herein, the term “diseased tissue” refers to a tissue sample or a tissue within an organism that has a disease. In one embodiment, the diseased tissue has an inflammatory liver disease or disorder. In one exemplary embodiment, the diseased tissue has non-alcoholic fatty liver disease (NAFLD) or non-alcoholic steatohepatitis (NASH). In another exemplary embodiment, the diseased tissue has alcoholic liver disease.

It should be understood that when the term “about” is used in the context of specific values or ranges of values, the disclosure should be read as to include the specific value or range referred to.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Various aspects of the invention are described in further detail in the following subsections.

### II. INFLAMMATORY LIVER DISEASES

The inflammatory response is an essential mechanism of defense of the organism against the attack of infectious agents, and it is also implicated in the pathogenesis of many acute and chronic diseases, including autoimmune disorders. In spite of being needed to fight pathogens, the effects of an inflammatory burst can be devastating. It is therefore often necessary to restrict the symptomatology of inflammation with the use of anti-inflammatory drugs. Inflammation is a complex process normally triggered by tissue injury that includes activation of a large array of enzymes, the increase in vascular permeability and extravasation of blood fluids, cell migration and release of chemical mediators, all aimed to both destroy and repair the injured tissue.

Inflammatory disorders of the liver (e.g., hepatitides) are prominent class of inflammatory disorders. Many of these disorders are associated with enhanced levels of inflammatory cytokines such as TNFα. For example, Alcoholic liver disease (ALD) is an inflammatory liver disorder associated with alcohol abuse or excessive or chronic ingestion of alcohol. Furthermore, alcohol intake induces liver damage and involves activation of the inflammatory cascade. Other inflammatory liver disorders include Non-alcoholic fatty liver disease (NAFLD) which is characterized by fatty inflammation of the liver that is not due to excessive alcohol use. Non-alcoholic steatohepatitis (NASH) refers to the most extreme form of NAFLD. Other inflammatory liver disorders or symptoms include liver cirrhosis, hepatocellular carcinoma (HCC), Biliary Atresia, Alagille Syndrome, Autoimmune Hepatitis, Alpha-1 Antitrypsin Deficiency (AlAD), Hemochromatosis, Wilson's disease, Tyrosinemia, Ischemic Hepatitis and Neonatal Hepatitis. In some embodiments, the inflammatory liver disorder is drug-induced hepatotoxicity. In other embodiments, the inflammatory liver disorder is viral hepatitis (e.g., Hepatitis A, B or C). In certain exemplary embodiments the liver disorder is alcoholic liver disease, non-alcoholic fatty liver disease (NAFLD) or non-alcoholic steatohepatitis (NASH).

As described herein, miR-155 overexpression in the liver is induced following chronic exposure to inflammatory stimuli such as ethanol and/or LPS stimulation. In particular, miR155 levels were shown to be upregulated in both macrophages and Kupffer cells. Surprisingly, the overexpression of miR-155 was correlated with increased TNFα mRNA stability in vivo, leading to increased release of TNFα. Thus, it is demonstrated herein that miR-155 plays a causative role in liver inflammation and contributes to the development of inflammatory liver disorders such as ALD. Accordingly, miR-155 is implicated as a link between the inflammatory response and inflammatory liver disorders. The data provided herein demonstrate the importance of proper regulation of miR-155, to avoid excessive activation of the inflammatory response and/or the development of inflammatory liver disease.

### III. MICRORNAS AND RNA SILENCING

MicroRNAs (miRNAs), also known as “small temporal RNAs” or “stRNAs”, are a group of naturally-occurring, single-stranded noncoding RNA molecules that regulate gene expression in eukaryotes by RNA silencing mechanisms. MicroRNAs are initially transcribed as long, single-stranded miRNA precursors known as a pri-miRNAs. These pri-miRNAs typically contain regions of localized stem-loop hairpin structures that in turn contain one or several embedded sequences of mature miRNAs. Pri-miRNAs are processed into 60-150 nucleotide pre-miRNAs in the nucleus by the double-stranded RNA-specific nuclease Drosha. These pre-miRNAs typically adopt a hairpin conformation with at least one stem-loop structure.

Pre-miRNAs are transported to the cytoplasm where the enzyme Dicer cleaves pre-miRNA to produce single-stranded mature miRNAs of about 20 to about 25 nucleotides (Hutvagner, 2002; McManus, 2002). Following processing, mature miRNAs are incorporated into an effector complex termed miRISC (miRNA-Induced Silencing Complex), which participates in RNA silencing. Canonical miRNAs influence gene expression by binding to sequences of partial complementarity in the 3′ UTR of mRNA and repressing their translation (McCaffrey, 2002). For example, miRNAs can pair with target mRNAs that contain sequences of only partial complementarity (e.g., 30%, 35%, 40%, 50%, 55%, 60%, 65%, 70%, 75%, 80% of sequence complementarity) to the miRNA. This is in contrast with siRNAs, which are of a similar size but are double-stranded RNA molecules having perfect or near-perfect complementarity to a target mRNA (e.g., 90% or more sequence complementarity), and operate via a RNAi cleavage mechanism. In recent studies, however, some miRNAs bearing perfect complementarity to a target RNA could function analogously to siRNAs, specifically directing degradation of the target sequences (Hutvagner, 2002b; Llave, 2002). Moreover, some miRNAs have been shown to activate up-regulate translation, instead of repressing it (see Vasudevan et al., Science, 2007, 318: 1931-1934).

### IV. miR155

As used herein, the term “miR155” refers to either mature (e.g., mature miR155) or precursor forms of miR155 (e.g., pre-miR155 or pri-miR155). Exemplary miR155 sequences are provided in Table 1 below. In a preferred embodiment, the miR155 is a human miR155 sequence, e.g., a human pri-miR155 (also known as the BIC transcript), human pre-miR155, or human mature miR155. Human pre-miR-155 (hsa miR-155; MirBase Accession No. MI0000681) was predicted based on homology to a cloned miR from mouse (mmu-miR-155; MiBase Accession No. MI0001777) [Lagos-Quintana M, et al., Curr Biol. 12:735-739 (2002)], and later experimentally validated in human HL-60 leukemia cells [Kasashima K, et al., Biochem Biophys Res Commun. 322:403-410 (2004)]. Like the mouse pri-miRNA155, human pri-miR-155 corresponds to a ˜1421 b.p. non-coding BIC transcript (EMBL:AF402776), located on chromosome 21 [Weber M J, FEBS J. 272:59-73 (2005)]. The mature form of human miR-155 (MIMAT0000646) differs from that in mouse at a single position. The “seed sequence” of human miR155 from positions three to eight, counting from the 5′ end of the mature miRNA sequence, is AAUGCU. The seed sequence of mouse miR155 is identical to that of human.

TABLE 1
Mir155 Sequences

| | |
|---|---|
| Human | 5′- |
| pre-miR-155 | CUGUUAAUGCUAAUCGUGAUAGGGGUUUUUGCCUCCAACUG |
| | ACUCCUACAUAUUAGCAUUAACAG-3′ |
| | 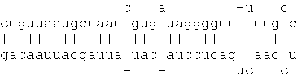 |
| Human | 5′-UUAAUGCUAAUCGUGAUAGGGGU-3′ |
| Mature | |
| miR-155 | |
| Mouse | 5′- |
| pre-miR-155 | CUGUUAAUGCUAAUUGUGAUAGGGGUUUUGGCCUCUGACUG |
| | ACUCCUACCUGUUAGCAUUAACAG-3′ |
| | 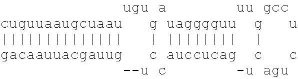 |
| Mouse | 5′-UUAAUGCUAAUUGUGAUAGGGGU-3′ |
| Mature | |
| miR-155 | |

Additional miR155 sequences can be found in miRBase, an online searchable database of miRNA sequences. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. It will be recognized by the skilled artisan that a miR-155 antagonist of the invention can be designed to target any version of miR-155, including the miR-155 sequences described in Release 10.0 of the miRBase sequence database and sequences described in any earlier or later releases of the miRBase sequence database which result in renaming or variations of a miR-155 sequence. Accordingly, the miR-155 antagonists of the present invention encompass modified oligonucleotides that are complementary to any sequence version of the miR-155 known in the art.

### V. miR155 ANTAGONISTS

As described herein, chronic inflammation of the liver (e.g., due to chronic ethanol exposure) increases miR155 in macrophages and Kuppfer cells and positively regulates the release of TNFα by enhancing its translation. The data provided herein also shows that the use of a miR155 antagonist can prevent the effects of miR-155 overexpression. Surprisingly and unexpectedly, the antagonism of miR155 resulted in only partial antagonism of TNF-α rather than complete inhibition. As used herein, the phrase “partial antagonism of TNF-α” refers to less than complete reduction in TNF-α protein expression levels (e.g., less than about 75%, about 50%, about 30%, about 20%, or less than about 10% reduction in TNFα expression levels). This is beneficial because low levels of TNF-α are required for immunity and complete knockdown of TNF-α or TNF-α activity (e.g., with anti-TNF-α antibodies) results in adverse affects on the animal. Thus, use of anti-miR155 antagonists according to the methods of the invention can have a beneficial effect on an animal suffering from an inflammatory liver disease, including for example, alcoholic liver disease, non-alcoholic fatty liver disease (NAFLD) or non-alcoholic steatohepatitis (NASH).

In one aspect, the invention provides miR155 antagonists for treating an inflammatory liver disease in a cell or a diseased tissue. A “miR155 antagonist”, as used herein, is an agent that reduces or inhibits the expression, stability, or activity of miR155. A miR155 antagonist may function, for example, by blocking miR155 activity (e.g., by blocking stabilization of TNFα mRNA by miR155). Additionally or alternatively, the miR155 antagonist may inhibit the biogenesis of miR155, for example, by blocking expression or processing of pre-miR155 or pri-miR155.

Exemplary miR155 antagonists are nucleic acid agents. These agents may include oligonucleotide antagonists, for example, antisense locked nucleic acid molecules (LNAs), antagomirs, or 2′O-methyl antisense RNAs targeting miR155. Other exemplary miR155 antagonists include anti-miR155 RNAi agents. In preferred embodiments, the miR155 antagonist compensates for the increase in miR155 and TNF-α that occurs during inflammatory liver disease (e.g., ALD). The effect of a miR155 antagonist on the level of miR155 and/or TNF-α in a cell can be determined by contacting the cell with a miR155 antagonist, and comparing the level of miR155 and/or TNF-α to a suitable control. In this embodiment, a preferred miR155 antagonist, or a preferred quantity of a miR155 antagonist, is one which decreases (e.g., by 30%, preferably 50% or more, more preferably 70% or more, still more preferably, 90% or more) the level of miR155 and/or TNF-α when compared to a suitable control, e.g., a comparable cell not contacted with a miR155 antagonist.

In certain embodiments, a miR-155 antagonist is a nucleic acid having a nucleotide sequence that is complementary to a miRNA-155 sequence, meaning that the nucleotide sequence is a least 60%, 65%, 70%, 75%, 80%, 85%, 90%, 95%, 97%, 98%, 99% or 100% identical to the complement of miR-155 precursor thereof over the entire length of the miRNA sequence or within a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleobases, or that the two sequences hybridize under stringent hybridization conditions. Accordingly, in certain embodiments the miR-155 antagonist may have one or more mismatched basepairs (e.g., 1, 2, 3, 4 or 5 mismatches) with respect to its target miR-155 sequence, and is capable of hybridizing to its target sequence. In certain embodiments, a miR-155 nucleic acid antagonist is perfectly complementary to miR-155 sequence. In certain embodiments, the nucleobase sequence of a modified oligonucleotide has full-length complementary to a miRNA.

(a) Anti-miR155 Antisense Oligonucleotides

In certain embodiments, a miR155 antagonist of the invention is an antisense oligonucleotide. The term “antisense” refers generally to an oligonucleotide (typically, a single-stranded oligonucleotide) that is sufficiently complementary to a target sequence to associate with the target sequence in a sequence-specific manner (e.g., hybridize to the target sequence). Exemplary antisense oligonucleotides in the instant application include oligoribonucleotide agents that hybridize to miR155 and block an activity/effect of the targeted RNA sequence, e.g., miR155 stabilization of TNFα mRNA.

Anti-miR155 antisense oligonucleotides may be rendered “nuclease resistant”. As used herein, the term “nuclease-resistant” refers to any modification which inhibits degradation by enzymes such as, for example, the exonucleases known to be present in the cytoplasm of a eukaryotic cell. RNA molecules (e.g., RNA oligonucleotides) are particularly at risk of degradation when combined with a composition comprising a cell extract or when introduced to a cell or organism, and a “ribonuclease-resistant” oligonucleotide is thus defined as an antisense molecule/agent that is relatively resistant to ribonuclease enzymes (e.g., endonucleases such as RISC), as compared to an unmodified form of the same oligonucleotide. Preferred antisense molecules/agents of the invention include those that have been modified to render the oligonucleotide relatively nuclease-resistant or ribonuclease-resistant. In a preferred embodiment, the antisense agents and/or oligonucleotides of the invention have been modified with a 2′-O-methyl group (e.g., 2′-O-methylcytidine, 2′-O-methylpseudouridine, 2′-methylguanosine, 2′-O-methyluridine, 2′-O-methyladenosine, 2′-O-methyl) and additionally comprise a phosphorothioate backbone.

In certain exemplary embodiments, the antisense anti-miR155 oligonucleotide is an antagomir. The term “antagomir,” as used herein, refers to small (e.g., 15-30 nucleotides, more preferably about 20-24 nucleotides) synthetic RNA-like oligonucleotides that are complementary to a specific miRNA target (i.e., miR-155), and that harbor various modifications for RNAse protection. Antagomirs differ from normal RNA by complete 2′-O-methylation of sugar, phosphorothioate backbone and a cholesterol-moiety at 3′-end. In some embodiments, antagomirs can have either mispairing at the cleavage site of Ago2, or a base modification at this site to inhibit Ago2 cleavage.

In other exemplary embodiments, the antisense anti-miR155 oligonucleotide is an LNA-oligonucleotide. The term “locked nucleic acid (LNA),” as used herein, refers to a nucleic acid analogue containing one or more LNA nucleotide monomers with a bicyclic furanose unit locked in an RNA mimicking sugar conformation. The ribose moiety of an LNA nucleotide is modified with an extra bridge (e.g., a 2′-O, 4′-C methylene bridge) connecting the 2′ and 4′ carbons. The bridge ‘locks’ the ribose in the 3′ endo structural conformation, which is often found in the A-form of RNA. LNA oligonucleotides display high hybridization affinity toward complementary single-stranded RNA, including miRNA. The locked ribose conformation of LNAs enhances base stacking and significantly increases the thermal stability of oligonucleotides containing LNAs.

In yet other exemplary embodiments, the anti-miRNA is a morpholino oligonucleotide. The terms “morpholinos” or “morpholino oligos,” as used herein, refers to nucleic acid analogs having standard nucleic acid bases that are bound to morpholine rings, rather than to deoxyribose rings, and are linked through phosphorodiamidate groups, rather than phosphates. Based on the similarity to natural nucleic acid structure, morpholinos bind to complementary sequences of mRNA by standard Watson-Crick base pairing. Instead of degrading their target RNA molecules, morpholinos act by steric blocking, binding to a target sequence within an RNA (e.g miR155) and inhibiting interaction of molecules which might otherwise interact with the RNA.

In order for certain antisense miRNA oligonucleotides to inhibit miRNA-155 as efficiently as possible there needs to be a certain degree of complementarity between the miRNA antagonist and miRNA-155. In some embodiments, it may be important for the oligonucleotide or antagomir to be complementary with the so-called “seed sequence” of a mature miRNA, e.g., positions 3 to 8, counting from the 5′ end of the mature miRNA. A miRNA “seed”, as used herein, refers to a region of about 6-8 contiguous nucleotides from the 5′ end of a miRNA having perfect or near perfect complementarity with about 6-8 contiguous nucleotides in a target RNA. In a preferred embodiment, a miRNA seed encompasses about nucleotides 2-7 (e.g., nucleotides 3-8, nucleotides 1-6, preferably nucleotides 2-7) from the 5′ end of a mature miRNA sequence. In exemplary embodiments, a miRNA seed has perfect complementarity with about 6-8 contiguous nucleotides in the 3′UTR of a target RNA. Nucleotide 1, counting from the 5′ end, is a non-pairing base and is most likely hidden in a binding pocket in the Ago 2 protein. Accordingly, the oligonucleotide or antagomir may or may not have a nucleotide in position 1, counting from the 3′ end, corresponding to nucleotide 1, counting from the 5′ end, of the corresponding target microRNA. In some cases, the first two nucleotides, counting from the 5′ end, of the corresponding target microRNA may be left unmatched.

In some embodiments, the core sequence of the anti-miR155 oligonucleotide is an RNA or DNA sequence from positions one to six, two to seven, or positions three to eight, counting from the 3′ end, and corresponding to positions three to eight, counting from the 5′ end, of miR155. In another embodiment, the miR155 antagomir oligonucleotide has a DNA or RNA sequence from positions one to seven, two to eight or three to nine, preferably from positions two to eight or three to nine, counting from the 3′ end. In another embodiment, the miR155 antagomir or oligonucleotide has a DNA or RNA sequence from positions one to eight, two to nine or three to ten, preferably from positions two to nine or three to ten, counting from the 3′ end, and corresponding to positions three to eight, counting from the 5′ end, of miR155. In yet another embodiment, the miR155 antagomir or oligonucleotide has a DNA or RNA sequence from positions one to nine, two to ten or three to eleven, preferably from positions two to ten or three to eleven, counting from the 3′ end.

The anti-miR155 oligonucleotides of the invention preferably have a total length of at least 6 nucleotides. For example, the oligonucleotides or antagomirs may have a total length of 6-15 nucleotides (6, 7, 8, 9, 10, 11, 12, 13, 14 or 15 nucleotides), 15-20 nucleotides (15, 16, 17, 18, or 19 nucleotides), 20-25 nucleotides (20, 21, 22, 23, 24 nucleotides), 25 or more nucleotides (25, 26, 27, 28, 29 nucleotides), or 30-35 nucleotides (30, 31, 32, 33, 34, 35 or more nucleotides). In certain embodiments, the oligonucleotide contains one or more repeats of the core sequence. In some embodiments, the core RNA or DNA sequence is substituted by a corresponding LNA sequence. For shorter oligonucleotides it may be necessary to increase the proportion of (high affinity) nucleotide analogues, such as LNA. Therefore in one embodiment at least about 30% of the nucleobases are nucleotide analogues, such as at least about 33%, such as at least about 40%, or at least about 50% or at least about 60%, such as at least about 66%, such as at least about 70%, such as at least about 80%, or at least about 90%. It will also be apparent that the oligonucleotide may comprise of a nucleobase sequence which consists of only nucleotide analogue sequences.

In certain embodiments, an anti-miR155 oligonucleotide comprises or consists of a number of linked nucleosides that is equal to the length of the miR-155 (e.g., the mature miR-155) to which it is complementary. In certain embodiments, the number of linked nucleosides of the oligonucleotide is less than the length of the miR-155 (e.g., mature miR-155) to which it is complementary (e.g., has one or two fewer nucleosides at its 5′ terminus and/or 3′ terminus). In certain embodiments, the number of linked nucleosides of an anti-miR155 oligonucleotide is greater than the length of the miRNA-155 (e.g., mature miR-155) to which it is complementary (e.g., has one or two more nucleosides at its 5′ terminus and/or 3′ terminus). In certain such embodiments, the nucleobase of an additional nucleoside is complementary to a nucleobase of a miRNA stem-loop sequence.

(b) Anti-miR155 RNAi Agents

In certain embodiments, the miR155 antagonist of the invention is an anti-miR155 RNAi agent. The term “RNAi agent”, as used herein, refers to an RNA (or analog thereof), having sufficient sequence complementarity to a target RNA (e.g., a target miRNA155) to direct RNAi of the target RNA. An RNAi agent having a “sequence sufficiently complementary to a target RNA sequence to direct RNAi” means that the RNA agent has a sequence sufficient to trigger the destruction of the target RNA by the RNAi machinery (e.g., the RISC complex) or process. Accordingly, RNAi agents can be designed to target any form of miR155 including a mature miR155, a pre-miR155, or a pri-miR155. For example, RNAi agents can be targeted to the stem and/or loop portions of a pre-miR155.

In one embodiment, an anti-miR155 RNAi agent of the invention is a siRNA. As used herein, the term “small interfering RNA” (“siRNA”) (also referred to in the art as “short interfering RNAs”) refers to an RNA agent, preferably a double-stranded agent, of about 10-50 nucleotides in length (the term “nucleotides” including nucleotide analogs), preferably between about 15-25 nucleotides in length, e.g., about 20-24 or 21-23 nucleotides in length, more preferably about 15, 16, 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference of a target RNA (e.g., miR155). The guide or antisense strand of the siRNA agent is designed to have a sequence sufficiently complementary to a miR155 sequence (e.g., a stem and/or loop sequence of a pre-miR155) to trigger RISC cleavage of the miR155 target RNA.

In another embodiment, an anti-miR155 agent is a shRNA. The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. shRNAs may be substrates for the enzyme Dicer, and the products of Dicer cleavage may participate in RNAi. In particular, embodiments, the shRNA can be designed to incorporate the sense and antisense sequences of a siRNA such that an siRNA is released from the shRNA following Dicer cleavage. shRNAs may be derived from transcription of an endogenous gene encoding a shRNA, or may be derived from transcription of an exogenous gene introduced into a cell or organism on a vector, e.g., a plasmid vector or a viral vector.

It will be apparent that anti-miR155 RNAi agents may also be modified to include one or more nucleotide analogs, e.g., modified RNAi agents with one or more modified nitrogeneous bases, sugar moeities and/or internucleotide linkage groups, provided that the activity of the RNAi agent is not adversely affected. Therefore in one embodiment at least about 30% of the nucleobases are nucleotide analogues, such as at least about 33%, such as at least about 40%, or at least about 50% or at least about 60%, such as at least about 66%, such as at least about 70%, such as at least about 80%, or at least about 90%.

In some embodiments, the foregoing miR155 antagonists can be expressed from an expression vector, e.g., a DNA vector or a viral vector. In preferred embodiments, the foregoing miR155 antagonists are expressed from a polymerase II or polymerase III promoter. In exemplary embodiments, an expression vector used to express a miR155 antagonist is a plasmid vector, an adenovirus vector, a lentivirus vector, or a YAC vector.

### VI. THERAPEUTIC APPLICATIONS

As described herein, the miR155 antagonists have therapeutic utility in the treatment of inflammatory liver disease (e.g., alcoholic liver disease). Accordingly, in one aspect, the invention provides methods for treating an animal, preferably a human, suspected of having or being prone to an inflammatory liver disease or condition, by administering a therapeutically or prophylactically effective amount of one or more of the miRNA antagonists of the invention, including any of the pharmaceutical compositions of the invention listed infra.

A cell or tissue that is contacted by a miR155 antagonist in accordance with the methods of the invention may be found within the animal. In this embodiment, administering a miR155 antagonist to the animal can inhibit inflammatory liver disease, for example, in a cell or tissue within the organism that is contacted by the miR155 antagonist.

In certain exemplary embodiments, the organism has alcoholic liver disease. In these embodiments, administering a miR155 antagonist to the organism is used to treat alcoholic liver disease (ALD). In other embodiments, the organism is at risk of developing ALD. In these embodiments, administering a miR155 antagonist to the organism is used to prevent alcoholic liver disease.

In other exemplary embodiments, the organism has non-alcoholic fatty liver disease (NAFLD). In these embodiments, administering a miR155 antagonist to the organism is used to treat NAFLD. In other embodiments, the organism is at risk of developing NAFLD or NASH. In these embodiments, administering a miR155 antagonist to the organism is used to prevent NAFLD.

In other exemplary embodiments, the organism has non-alcoholic steatohepatitis (NASH). In these embodiments, administering a miR155 antagonist to the animal is used to treat NASH. In other embodiments, the animal is at risk of developing NASH. In these embodiments, administering a miR155 antagonist to the animal is used to prevent NASH.

It will be recognized by those of skill in the art that miR155 antagonists may also be used for the treatment or prevention of other inflammatory liver disorders, including without limitation hepatitis, liver cirrhosis, hepatocarcinoma, Biliary Atresia, Alagille Syndrome, Alpha-1 Antitrypsin, Tyrosinemia, Neonatal Hepatitis and Wilson Disease.

As will be understood, dosing is dependent on severity and responsiveness of the disease state to be treated, and the course of treatment lasting from several days to several months, or until a cure is effected or a diminution of the disease state is achieved. Optimal dosing schedules can be calculated from measurements of drug accumulation in the body of the patient. Optimum dosages may vary depending on the relative potency of individual oligonucleotides. Generally it can be estimated based on EC50s found to be effective in in vitro and in vivo animal models. In general, dosage is from 0.01 μg to 1 g per kg of body weight, and may be given once or more daily, weekly, monthly or yearly, or even once every 2 to 10 years or by continuous infusion for hours up to several months. The repetition rates for dosing can be estimated based on measured residence times and concentrations of the drug in bodily fluids or tissues. Following successful treatment, it may be desirable to have the patient undergo maintenance therapy to prevent the recurrence of the disease state.

### VII. DIAGNOSTIC APPLICATIONS

The miRNA antagonists of the present invention can be utilized for use as a diagnostic reagent. In diagnostics the oligonucleotides may be used to detect and quantitate target expression in cell and tissues by Northern blotting, in-situ hybridisation or similar techniques. For therapeutics, an animal or a human, suspected of having an inflammatory liver disease or disorder, can be diagnosed by administering the miRNA antagonist of the invention (e.g., anti-miR155 oligonucleotides or antagomirs) and detecting the level of bound product.

In one embodiment of the invention, a liver inflammatory disorder is diagnosed in an animal by assaying a liver sample isolated from the animal to determine the amount of miR-122 and/or miR-155 in the sample, and diagnosing a liver inflammatory disorder in the animal if the amount of miR-122 and/or miR-155 in the liver sample is higher than the amount of miR-122 and/or miR-155 in normal liver tissue. In another embodiment of the invention, a liver inflammatory disorder is diagnosed in an animal by determining the amount of miR 122 and/or miR-155 in a liver sample isolated from the animal; determining the amount of miR-122 and/or miR-155 in normal liver cells; and diagnosing a liver inflammatory disorder in the animal if the amount of miR-122 and/or miR-155 in the liver sample isolated from the animal is higher than the amount of miR-122 and/or miR-155 in the normal liver-cells. In preferred embodiments of the invention, the liver disorder being diagnosed in the animal is liver inflammation, liver damage, alcoholic liver disorder (ALD), non-alcoholic fatty liver disease (NAFLD) or non-alcoholic steatohepatitis (NASH). In one embodiment, elevated levels of miR-122 and/or miR-155 are used to diagnose a liver disorder in its early stages (i.e., before progression to cancer) or to predict the propensity of a subject to develop a liver disorder. In another embodiment, elevated levels of miR-122 and/or miR-155 can be used to detect liver damage or disease not caused by alcohol. In one embodiment, elevated levels of miR-155 are used to diagnose or predict liver inflammation. In another embodiment, elevated levels of miR-122 are used to diagnose or predict hepatocyte damage. In preferred embodiments of the invention, the animal being diagnosed is a mammal, and even more preferably, the animal being diagnosed is a human.

Biological samples from a subject can be taken using methods known to those in the are and can comprise cells (e.g., from a liver biopsy) or can comprise bodily fluids (e.g., serum or plasma).

In the methods of the invention, the amount of miR-122 and/or miR-155 in a given sample may be determined using any suitable procedure for quantitating RNA known to those of skill in the art, including, but not limited to, polymerase chain reaction (PCR), ligase chain reaction (LCR), self-sustained sequence replication system (Guatelli et al., 1990, Proc. Natl. Acad. Sci. U.S.A. 87:1874-78), Q-beta replicase method, Northern blot assay, RNase protection assay, cycling probe reaction (Duck et al., 1990, Biotechniques 9:142-48), and branched DNA (bDNA) method (Urdea et al., 1987, Gene 61:253-64). In preferred embodiments of the invention, invasive cleavage reactions (U.S. Pat. No. 6,692,917; E is et al., 2001, supra; and U.S. Patent Application Publication Nos. 2003/0104378 and US 2003/0186238)—such as those sold under the trademark Invader®—are used to determine the amount of miR-122 and/or miR-155 in a given sample.

In one method of the invention, normal (or control) may be obtained from a healthy individual. In another method of the invention, normal (or control) may be obtained from a cultured cell line, provided that the cultured cell line expresses an amount of miR-122 and/or miR-155 that is comparable to that expressed by liver cells isolated from a healthy individual. In one method of the invention, the amount of miR-122 and/or miR-155 in normal cells is determined by referring to a reference standard for the amount of miR-122 and/or miR-155 expression for normal cells (for example, compiled from a plurality of normal individual liver samples), or that is otherwise known or can be readily determined by those of skill in the art.

In a preferred embodiment of the invention, the diagnosis of inflammatory liver disorder is based on an observation that the amount of miR-122 and/or miR-155 in the liver sample isolated from the animal is at least two times higher (e.g., at least 5 times higher, at least 10 times higher, at least 15 times higher, or at least 20 times higher) than the amount of miR-122 and/or miR-155 in the normal liver cells. In other preferred embodiments, the diagnosis of inflammatory liver disorder is based on an observation that an observed increase of miR-155 in the liver sample isolated from the animal correlates with an observed increase and/or stability of TNFα mRNA in the sample. In other preferred embodiments, the diagnosis of inflammatory liver disorder is based on an observation that an observed increase of miR-122 in the liver sample isolated from the animal correlates with an observed increase in ALT (alanine aminotransferase) in the sample.

### VIII. SCREENING APPLICATIONS

The invention also provides methods for identifying compounds for the treatment of inflammatory liver disorders. In one embodiment of the invention, a compound for treating an inflammatory liver disorder is identified by determining the amount of miR-155 in a cell sample (e.g., a sample of hepatocytes, Kuppfer cells or macrophages); exposing the sample to the compound; determining the amount of miR-155 in the sample following exposure of the sample to the compound; and identifying a compound for treating the inflammatory liver disorder if the amount of miR-155 in the liver sample before exposure to the compound is higher than the amount of miR-155 in the liver sample after exposure to the compound. In one method of the invention, the sample is obtained from a cultured cell line, or from one or a plurality of clinical samples.

In a preferred embodiment of the invention, the identification of a suitable compound for treating an inflammatory liver disorder is based on an observation that the amount of miR-155 in the sample before exposure to the compounds is at least two times higher (e.g., at least 5 times higher, at least 10 times higher, at least 15 times higher, or at least 20 times higher) than the amount of miR-155 in the sample after exposure to the compound. In other preferred embodiments, the identification of a suitable compound is based on an observed decrease in the level and/or stability of TNFα mRNA in the sample.

### IX. PHARMACEUTICAL COMPOSITIONS

In another aspect, the invention provides pharmaceutical compositions comprising a miR155 antagonist and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, intraperitoneal, intramuscular, oral (e.g., inhalation), transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the miR155 antagonist can be incorporated with excipients and used in the form of tablets, troches, or capsules, e.g., gelatin capsules. Pharmaceutically compatible binding agents, and/or adjuvant materials can be included as part of the composition.

For administration by inhalation, the miR155 antagonist may be delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the miRNA antagonist may be formulated into ointments, salves, gels, or creams as generally known in the art.

The miR155 antagonist can also be administered by transfection or infection using methods known in the art, including but not limited to the methods described in McCaffrey et al. (2002), Nature, 418(6893), 38-9 (hydrodynamic transfection); Xia et al. (2002), Nature Biotechnol., 20(10), 1006-10 (viral-mediated delivery); or Putnam (1996), Am. J. Health Syst. Pharm. 53(2), 151-160, erratum at Am. J. Health Syst. Pharm. 53(3), 325 (1996).

In some embodiments, the miR155 antagonists and RNA silencing agents (and other optional pharmacological agents) can be delivered directly via a pump device. For example, in some embodiments, the miR155 antagonists or RNA silencing agents of the invention are delivered directly by infusion into the diseased tissue, e.g. a tissue or cells that have alcoholic liver disease.

The miR-155 antagonists can also be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al. (1998), Clin. Immunol. Immunopathol., 88(2), 205-10. Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the miR155 antagonists are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

In one embodiment, the miRNA155 antagonists are formulated in the form of a yeast cell wall particle (YCWP, e.g., as described in US Patent Publication No. 20090226528, which is incorporated herein by reference in its entirety). YCWP are hollow and porous 2-4 micron microspheres prepared from yeast, for example, Baker's yeast, composed primarily of beta 1,3-D-glucan and, optionally, chitin and/or mannoprotein. Briefly, the process for producing the YCWPs involves the extraction and purification of the alkali-insoluble glucan particles from the yeast or fungal cell walls. Preparation of glucan particles involves treating the yeast with an aqueous alkaline solution at a suitable concentration to solubilize a portion of the yeast and form an alkali-hydroxide insoluble glucan particles having primarily .beta.(1,6) and .beta.(1,3) linkages. The alkali generally employed is an alkali-metal hydroxide, such as sodium or potassium hydroxide or an equivalent. The intracellular components and, optionally, the mannan portion, of the cell are solubilized in the aqueous hydroxide solution, leaving insoluble cell wall material which is substantially devoid of protein and having substantially unaltered .beta.(1,6) and .beta.(1,3) linked glucan. The intracellular constituents are hydrolyzed and released into the soluble phase. The conditions of digestion are such that at least in a major portion of the cells, the three dimensional matrix structure of the cell walls is not destroyed. In particular circumstances, substantially all the cell wall glucan remains unaltered and intact.

YCWPs can be used to deliver a payload of encapsulated miR155 antagonist to a cell. In some embodiments, the miR155 antagonist is complexed with polyelectrolyte trapping agent to form nanoparticle that is caged within the YCWP. Formation of the YCWP encapsulated polyelectrolyte nanoparticles follows a layer-by-layer (LbL) approach, with the different components assembled through electrostatic interactions. In some embodiments, the nanoparticle is formed around a core comprising an inert nucleic acid, such as tRNA or scrambled RNA, and a trapping agent. Other exemplary core components include, but are not limited to, anionic polysaccharides, proteins, synthetic polymers and inorganic matrices. Exemplary trapping agents are cationic polyeletrolytes and can include, but are not limited to, cationic polysaccharides, proteins and synthetic polymers. Exemplary YCWPs feature layers comprising a trapping molecule for the payload, which can be a cationic agent, such as an agent used to prepare nucleic acids for transfection into cells (e.g., polyethylenimine (PEI)); an inert nucleic acid, such as tRNA; and miR155 antagonist as a payload molecule. In certain exemplary embodiments, up to 100 mu.g of payload miR155 antagonist is added per 1.times.10.sup.9 YCWP with tRNA/PEI cores. A trapping molecule (e.g., PEI) is then added at a trapping molecule/nucleic acid ratio of 2.5 to coat the nucleic acid/tRNA-PEI core. Alternative embodiments include nanocomplexes and nanoparticles wherein a trapping molecule/layer is not applied to the YCWP/tRNA-PEI core/payload nucleic acid complex. Other embodiments include nanocomplexes and nanoparticles wherein a payload molecule is incorporate directly into the core, with or without tRNA.

Toxicity and therapeutic efficacy of miR155 antagonists can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects. The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the compound which achieves a half-maximal inhibition of symptoms) as determined in cell culture. Such information can be used to more accurately determine useful doses in humans. Levels in plasma may be measured, for example, by high performance liquid chromatography.

A therapeutically effective amount of a pharmaceutical composition containing a miRNA antagonist of the invention (i.e., an effective dosage) is an amount that inhibits expression and/or activity of miR155 and/or TNF-α by at least 10 percent, more preferably at least 30%. Higher percentages of inhibition, e.g., 40, 50, 75, 85, 90 percent or higher may be preferred in certain embodiments. Exemplary doses include milligram or microgram amounts of the molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram.

In certain embodiments, pharmaceutical compositions comprise a miR155 antagonist at a dose selected from 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 270 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, and 800 mg. In certain such embodiments, a pharmaceutical composition of the present invention comprises a dose of miR-155 antagonist selected from 25 mg, 50 mg, 75 mg, 100 mg, 150 mg, 200 mg, 250 mg, 300 mg, 350 mg, 400 mg, 500 mg, 600 mg, 700 mg, and 800 mg.

The compositions can be administered one time per week for between about 1 to 10 weeks, e.g., between 2 to 8 weeks, or between about 3 to 7 weeks, or for about 4, 5, or 6 weeks. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a composition can include a single treatment or a series of treatments.

It is furthermore understood that appropriate doses of a composition depend upon the potency of composition with respect to the expression or activity to be modulated. When one or more of these molecules is to be administered to an animal (e.g., a human) to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

This invention is further illustrated by the following examples which should not be construed as limiting. The contents of all references, patents and published patent applications cited throughout this application are incorporated herein by reference in their entirety.

### EXAMPLES

### Materials and Methods

In general, the practice of the present invention employs, unless otherwise indicated, conventional techniques of chemistry, molecular biology, and recombinant DNA technology. See, e.g., Sambrook, Fritsch and Maniatis, Molecular Cloning: Cold Spring Harbor Laboratory Press (1989); and Current Protocols in Molecular Biology, eds. Ausubel et al., John Wiley & Sons (1992).

A. Animal Studies and Kuppfer Cell (KC) isolation. Four-week-old female mice (C57BL/6) were divided into two groups (15 mice per group). Ethanol-fed group received the Lieber-DeCarli diet (Bio-Serv, Frenchtown, N.J.) with 4.5% (volume/volume) ethanol (32.4% ethanol-derived calories) for 4 weeks; pair-fed control mice received an equal amount of calories as their alcohol-fed counterparts with the alcohol-derived calories substituted with dextran-maltose. Mice were bled by submandibular venipuncture and serum was separated from whole blood and frozen at −80° C. In four mice, livers were fixed in formalin and were further paraffin embedded, sectioned, and stained with hematoxylin-eosin for microscopic analysis. The rest of the mice received anesthesia with ketamine (100 mg/kg) and KCs were isolated as previously described (Hritz et al., Hepatology, 2008, 48: 1224-1231). Briefly, the livers were perfused with saline solution for 10 minutes followed by in vivo digestion with liberase enzyme for 5 minutes and in vitro digestion for 30 minutes. The nonhepatocyte content was separated by Percoll gradient and centrifuged for 60 minutes at 800 g. The intercushion fraction was washed and adhered to plastic in Dulbecco's modified Eagle's medium+5% fetal bovine serum. The nonadherent fraction was washed and the adherent KC population was adjusted to 2×106/mL in Dulbecco's modified Eagle's medium+10% fetal bovine serum. Cells from 2-3 mice were pooled for each experiment given the limited number of KCs available from each animal.

B. Biochemical Assays. Serum alanine aminotransferase (ALT) activity was determined using a kinetic method (Advanced Diagnostics Inc., South Plainfield, N.J.), serum endotoxin levels were measured using the Limulus amebocyte lysate assay (Lonza Walkersville Inc, Walkersville, Md.) and serum alcohol levels were determined using an alcohol analyzer (Analox Lunenberg, Mass.). TNF-α was estimated in cell-free supernatants using ELISA from BD Pharmingen (San Diego, Calif.).

C. Cell culture and reagents. RAW 264.7 macrophages were purchased from American Type Culture Collection and maintained in Dulbecco's modified medium (Invitrogen Life Technologies, Carlsbad, Calif.) containing 10% FBS (HyClone, South Logan, Utah) at 37° C. in a 5% CO2 atmosphere. For prolonged alcohol exposure, cells exposed to 50 mM alcohol were placed in a Billups-Rothenburg chamber with twice the alcohol concentration in the bottom of the chamber to saturate the chamber and maintain a stable alcohol concentration, as previously described (Romics et al., Hepatology, 2004, 40: 376-385). Actinomycin D and MG-132 was purchased from Sigma-Aldrich (St. Louis, Mo.). LPS (Echerichia coli strain 0111:B4) was from Difco (Detroit, Mich.). KCs and RAW 264.7 macrophages were stimulated with Escherichia coli-derived LPS (100 ng/ml), 50 mM ethanol, or the combination of LPS and ethanol at the times indicated in the figure legends.

D. Transfection. For inhibition of miR155, RAW 264.7 macrophages were transfected with anti-miR155 and anti-miR control and for overexpression of this miRNA RAW cells were treated with pre-miR-155 and Pre-miR Precursor Negative Control #1 using siPORT NeoFx transfection agent. All reagents were purchased from Ambion Inc. (Austin, Tex.). Knock-down efficiency was determined by transfecting the cells with GAPDH siRNA (Ambion) and overexpression efficiency was checked by determining miR155 levels in transfected cells. Transfected cells were treated or not with 50 mM ethanol for 48 h; stimulated or not with LPS (100 ng/ml) and treated or not with actinomycin D according to experimental requirements, before the isolation of RNA or supernatant collection.

E. RNA analysis. RNA was purified using the RNeasy kit (Qiagen Sciences, Maryland, USA) or mirVana™ miRNA Isolation Kit (Ambion) if miRs were to be analyzed. The quality of RNA was routinely checked by measurement of OD (260/280 and 260/230 ratio) and gel electrophoresis. Quantitative RT-PCR analyses for miR-125b, miR-146aa, miR155 and sno202, used as normalizing control, were performed using TaqMan miRNA assays with reagents, primers, and probes obtained from Ambion. In brief, a stem loop primer was used for reverse transcription (30 min, 16° C.; 30 min, 42° C.; 5 min 85° C.), followed by qPCR employing FAM-TaqMan probes and primers in an Eppendorf Realplex Mastercycler (Eppendorf, Westbury, N.Y.). For TNF-α and 18s mRNA expression, RNA was cDNA transcribed with the Reverse Transcription System (Promega Corp., Madison, Wis.). Real-time quantitative polymerase chain reaction was performed using the iCycler (Bio-Rad Laboratories Inc., Hercules, Calif.), as described previously (Romics et al., supra). The primer sequences were as follows: TNF-α, forward 5′-GTA ACC CGT TGA ACC CCA TT-3′ and reverse 5′-CCA TCC AAT CGG TAG TAG CG-3′; 18s, forward 5′-CAC CAC CAT CAA GGA CTC AA-3′ and reverse 5′-AGG CAA CCT GAC CAC TCT CC-3′. Relative expression was calculated using the comparative threshold cycle (Ct) method.

F. Statistical Analysis. Data are presented as mean±SE and groups were compared by means of Student's t-test or Mann Whitney-U test according to data distribution. Correlation was assessed by means of Spearman's rho test. P<0.05 was regarded as significant.

### Example 1

### MiR155 Expression is Up-Regulated in Macrophages after Ethanol and/or LPS Stimulation In Vitro and Correlates with TNF-α Production

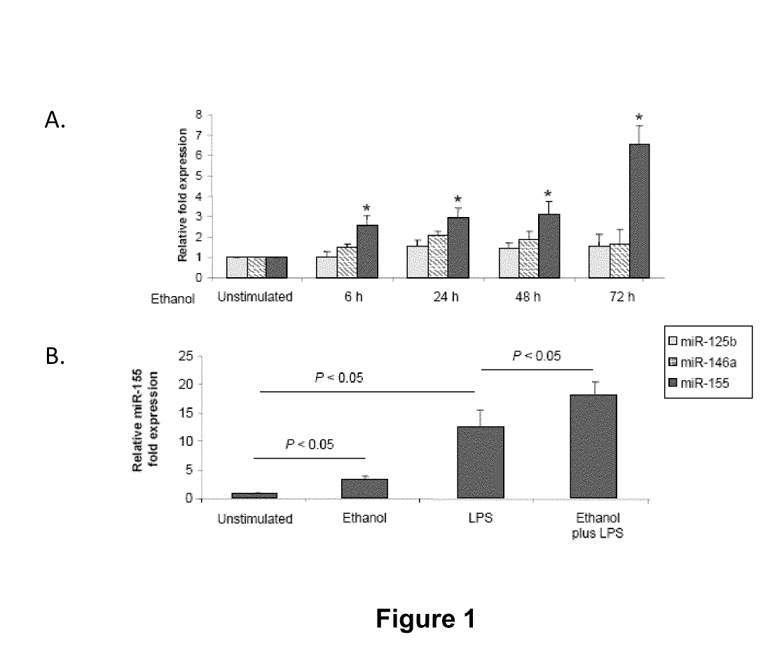

TNF-α, an LPS-induced cytokine, is increased in ALD (Mandrekar & Szabo, J. Hepatol., 2009, 50: 1258-1266). In particular, prolonged ethanol exposure leads to an increase in inflammatory cell responses, particularly in LPS-induced TNF-α production in macrophages and Kupffer cells (KCs). To test whether alcohol affects TNF-α production via regulation of miRNAs, RAW 264.7 cells, a surrogate model of KCs with respect to alcohol-induced TNF-α production (Szabo & Mandrakar, Alcohol Clin. Exp. Res., 2009, 33: 220-232), were studied. It was determined that a physiologically relevant dose of ethanol (50 mM) resulted in significant up-regulation of miR155 within 6-72 hours, with the highest induction in the presence of prolonged alcohol exposure (72 hours) (FIG. 1A). Notably, the alcohol-induced increase was specific to miR-155 as there were no significant changes in miR-146a or miR-125b after alcohol treatment (FIG. 1A). Addition of LPS, a ligand of the Toll-like receptor TLR4, also resulted in a significant increase in miR155 expression in RAW cells (FIG. 1B). More importantly, ethanol pretreatment augmented the LPS-induced increase in miR155 levels (FIG. 1B). Thus, these data suggested that miR155 is involved in ethanol-induced changes in macrophage activation.

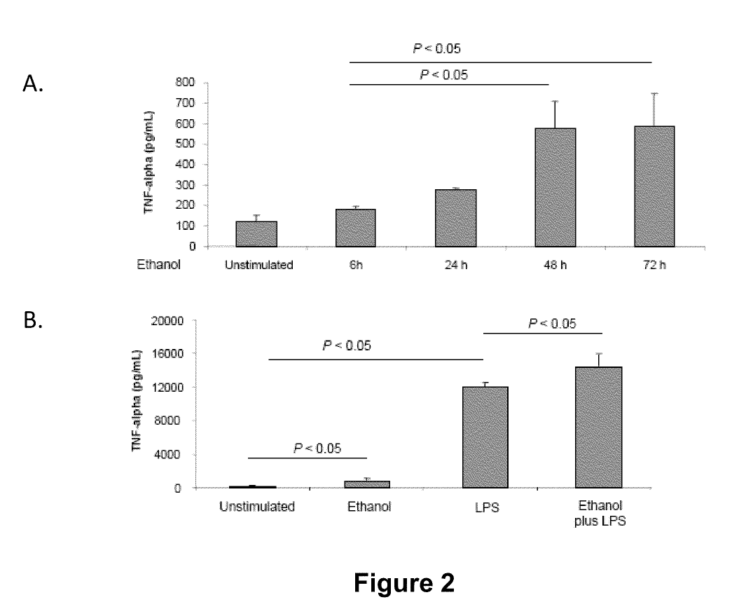

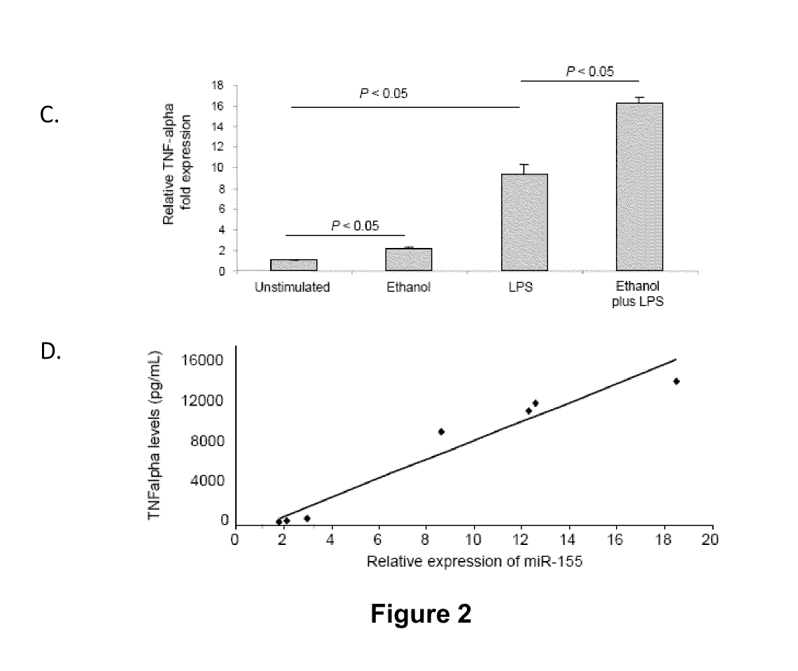

Examination of TNF-α production in RAW cells also revealed a time-dependent induction of TNF-α (FIG. 2A). Furthermore, prolonged pretreatment of RAW cells with ethanol augmented LPS-induced TNF-α production (FIGS. 2B and 2C). Changes in TNF-α production and miR155 expression were parallelled in macrophages. Indeed, a significant correlation (R=0.94) existed between TNF-α levels and miR155 expression after ethanol and/or LPS stimulation (FIG. 2D). These results further demonstrate that that miR155 functions in LPS-induced TNF-α production in alcohol treated macrophages. Further experiments were done and the statistics obtained were recalculated (see Bala et al. J. Biol. Chem. 2011. 286:1436-1444) incorporated herein by this reference.

### Example 2

### MiR155 is Up-Regulated In Vivo in Kupffer Cells of Alcohol-Fed Mice

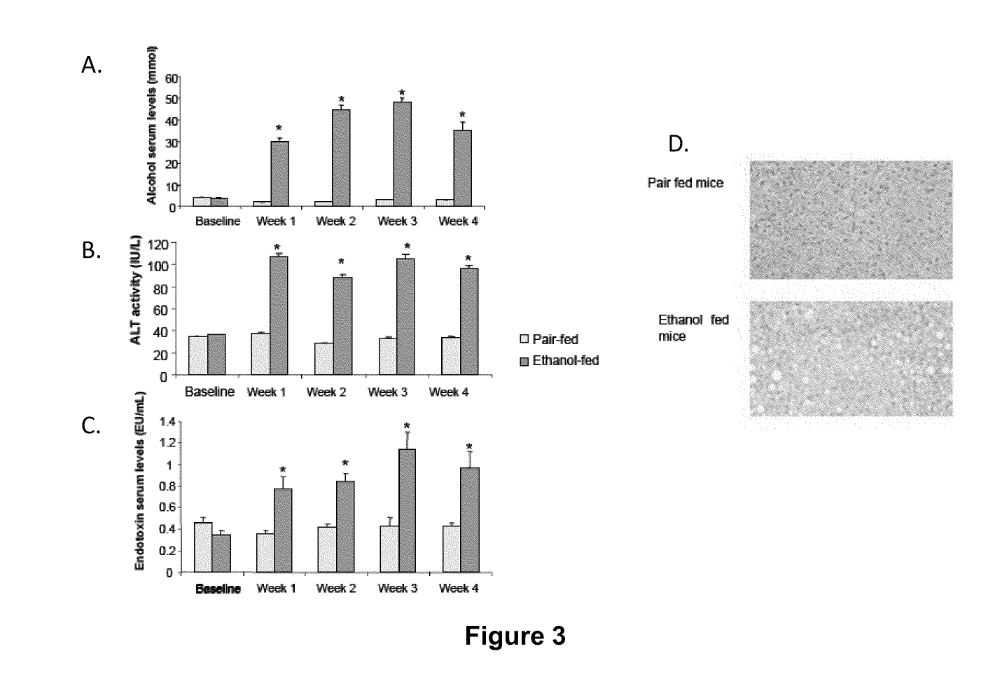

Chronic alcohol feeding of mice with a Lieber-DeCarli diet results in a significant increase in serum ALT, serum ethanol and endotoxin levels in mice as early as 1 week after alcohol feeding. These abnormalities are sustained throughout the 4 weeks of alcohol feeding (FIG. 3A, B and C). Further experiments also included measurements at weeks 2 and 3 (see Bala et al. J. Biol. Chem. 2011. 286:1436-1444) incorporated herein by this reference. Evaluation of liver histology reveals the presence of steatosis and inflammatory cells in ethanol-fed (but not pair-fed) mice (FIG. 3D).

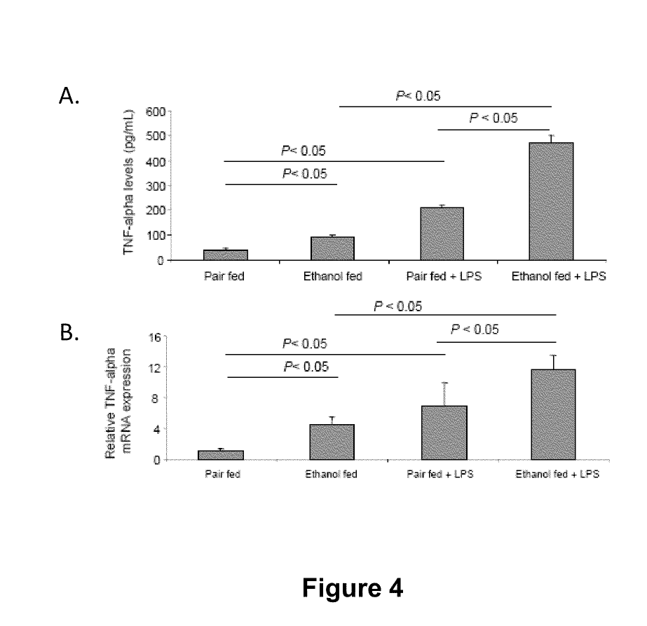

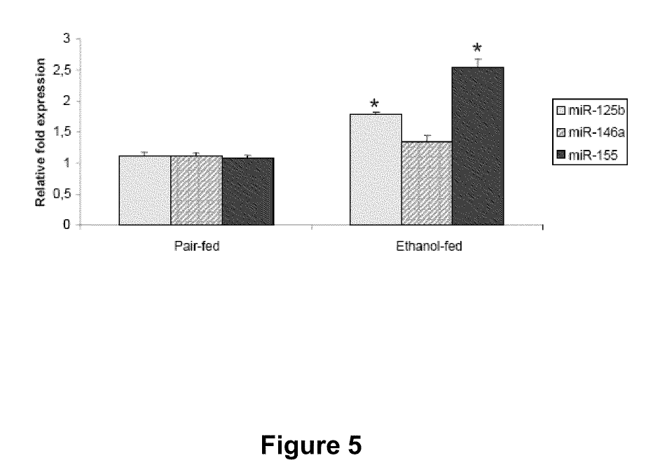

To assess the in vivo effects of alcohol on miR155 and TNF-α production, Kupffer cells were isolated from livers of alcohol-fed and control mice. KCs isolated from ethanol-fed mice showed increased TNF-α production and TNF-α mRNA expression compared to pair-fed mice (FIG. 4 A, B). Further experiments were done and the statistics obtained were recalculated (see Bala et al. J. Biol. Chem. 2011. 286:1436-1444) incorporated herein by this reference. LPS stimulation in KCs from ethanol-fed mice resulted in significantly higher TNF-α production both at protein and mRNA levels compared to KCs from pair-fed mice (FIG. 4). Consistent with the in vitro effects of prolonged alcohol, there was a significant increase in miR155 expression in KCs from ethanol-fed mice (FIG. 5). Interestingly, the level of miR-125b but not miR-146a was also increased after alcohol feeding in KCs. Further experiments demonstrated that in vitro alcohol exposure showed no significant effect on miR-155 levels in KCs from alcohol-fed mice with or without in vitro LPS stimulation. A similar pattern was seen when TNFα levels were measured (see Bala et al. J. Biol. Chem. 2011. 286:1436-1444) incorporated herein by this reference. These data demonstrate for the first time that miR155 up-regulation occurs in vivo in KCs after chronic ethanol intake in the liver.

### Example 3

### MiR155 Expression is Unregulated in Total Livers and Hepatocytes after Exposure to Alcohol

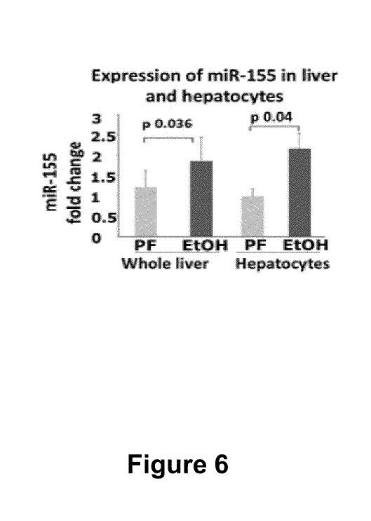

The role of miR-155 was also elucidated in different cell types of the liver. After mice were treated for 4 weeks with the Lieber-DeCarli diet (liquid alcohol diet), increased expression of miR-155 was observed in total livers and in isolated hepatocytes from the alcohol-fed mice (FIG. 6). Together with the observation that prolonged alcohol exposure increases miR-155 in macrophages and Kupffer cells, this observation provides that miR-155 is also increased in hepatocytes in alcoholic liver disease. Thus, these data demonstrate that miR-155 is involved in cell-specific functions.

### Example 4

### MiR155 Enhances LPS-Induced TNF-α Production after Chronic Ethanol Exposure by Increasing TNF-α mRNA Stability

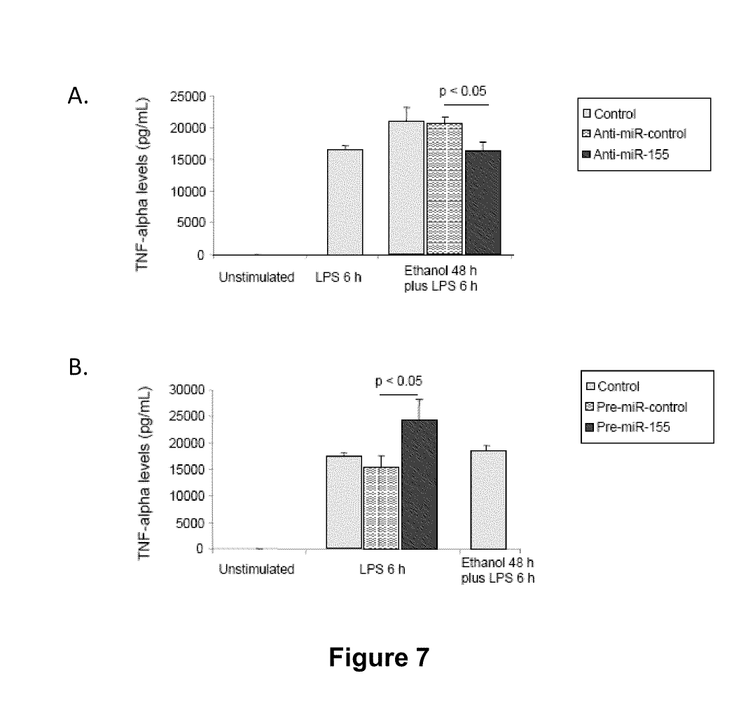

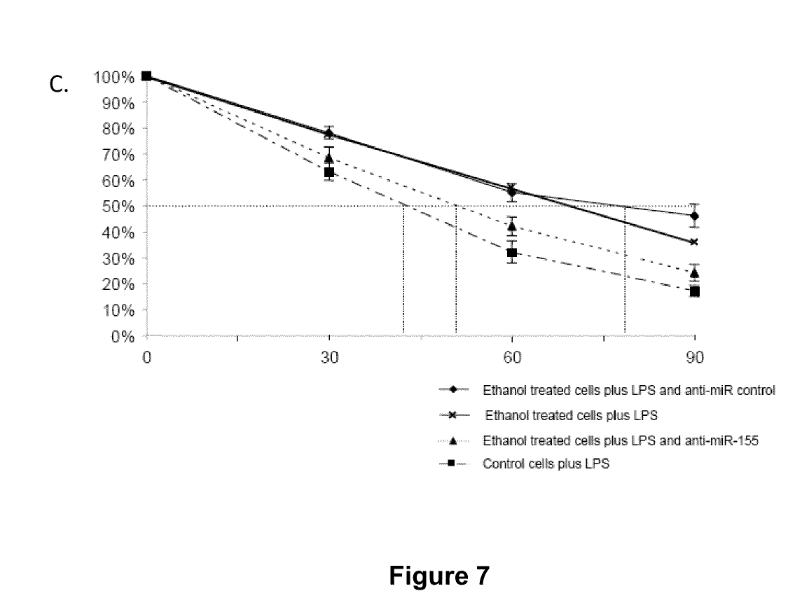

To evaluate a causative relationship between the alcohol-induced increase in miR155 and increased TNF-α levels, an inhibitor of miR155 was used. Inhibition of miR155 resulted in a significant decrease in LPS-induced TNF-α production both in alcohol-treated and alcohol-naïve cells (FIG. 7A). To further evaluate the effect of miR155 overexpression, RAW cells were transfected with a pre-miR-155 precursor. The premiR-155, but not the pre-miR control, resulted in a significant increase in TNF-α production after LPS stimulation in alcohol-treated and in alcohol-naive cells (FIG. 7B). Further experiments which further confirm these findings (see Bala et al. J. Biol. Chem. 2011. 286:1436-1444) incorporated herein by this reference. Furthermore, inhibition of miR155 with an anti-miR-155 oligonucleotide reduced LPS-induced TNF-α mRNA half life (˜50 minutes) in RAW cells exposed to ethanol compared treatment with anti-miR-control (˜75 minutes) (FIG. 7C). Further experiments were done which further confirm these findings (see Bala et al. J. Biol. Chem. 2011. 286:1436-1444) incorporated herein by this reference. These data indicate that miR155 increases LPS-induced TNF-α production by means of enhancing mRNA stability in alcohol-treated RAW cells.

### Example 5

### Nuclear Factor-Kappa B (NF-κB) Inhibition Prevented MiR155 Increase after LPS and/or Ethanol Exposure

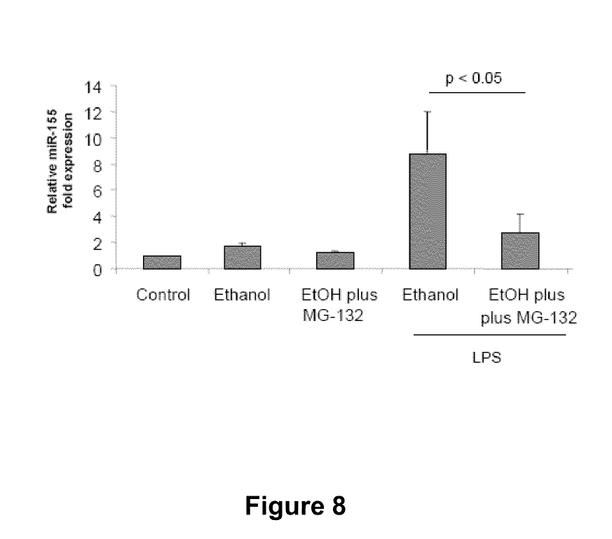

It was further investigated whether inhibition by MG-132 or Bay 11-7082, which block NF-κB activation through inhibiting proteosome degradation of IκB (Qureshi et al., J. Immunol., 2003, 171: 1515-1525), could block miR155 increase after LPS stimulation or ethanol exposure. To analyze this, RAW cells were pretreated for 48 hours with ethanol (50 mM) and MG-132 (0.25 μM) or Bay 11-7082 (0.1 uM). As shown in FIG. 8, MG-132 or Bay 11-7082 pretreatment resulted in a decrease of miR155 levels after ethanol and/or LPS stimulation. Further experiments were done which further confirm these findings (see Bala et al. J. Biol. Chem. 2011. 286:1436-1444) incorporated herein by this reference. These experiments suggest a functional role for NF-kB activation in miR-155 up-regulation by alcohol in KCs.

### Example 6

### Serum MiR122 and MiR155 as Biomarkers of Liver Injury and Inflammation

It has been determined that miRs expression levels change not only in diseased tissues but also in serum or plasma. In addition, because miRs are stable in frozen samples, they are attractive for biomarker discovery.

The importance of serum/plasma miRs, prior to this invention, had not been explored in liver disease. Micro-RNA122 is expressed in high abundance in hepatocytes where it regulates different metabolic pathways while miR-155 is a central regulator of inflammation. Evaluation of the serum levels of miRs as potential markers of hepatocyte damage (miR-122) and inflammation (miR-155) was assessed in experimental models of liver injury induced by alcohol, carbon tetrachloride (CCL4), or acetaminophen (APAP).

To evaluate miRs as biomarkers of liver injury and inflammation, serum/plasma and liver samples were collected from C57/B16 (WT) mice after: 1. Chronic alcohol feeding with a Lieber-DeCarli or control pair-fed diet at 1-4 weeks; 2. CCL4 administration for 2-6 weeks; 3. APAP (500 mg/kg) injection for 0.5-6 hours. Serum alanine aminotrasferase (ALT) was evaluated and total RNA was analyzed for miR-122 and miR-155 expression with Taq Man MicroRNA assay using miR-16 and miR-223 as housekeeping controls. Student two-tailed T test was used for statistics.

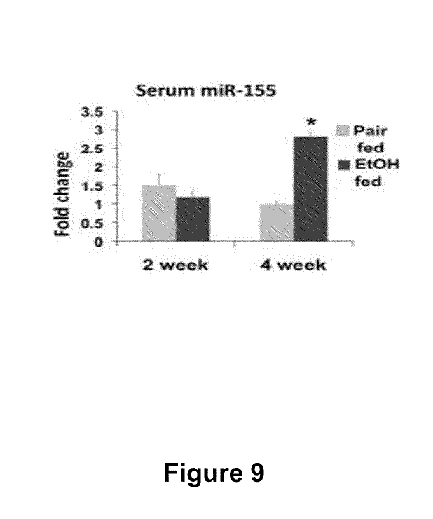

The alcohol-, CCL4-, and APAP-induced liver injury models all resulted in a significant increase in ALT and more important, in increased serum/plasma miR-122 levels compared to control WT mice. After alcohol or CCL4 treatment, serum miR-122 was up-regulated as early as one week over controls and it remained elevated. There was a linear correlation between serum miR-122 and ALT levels (Pearson test p<0.05). In contrast, there was no increase in serum miR-122 in Toll like receptor 4 (TLR4 KO) or NADPH oxidase-deficient (p47^{phox} KO) mice after alcohol feeding as these KO mice were protected from alcohol-induced liver injury, steatosis and inflammation. In the APAP model in WT mice, a significant increase in serum miR-122 levels was observed before ALT elevations suggesting that serum miR-122 might be an early biomarker of liver injury. Alcohol-, CCL4-, and APAP-induced liver damage all involve activation of the inflammatory cascade. Consistent with this, increased serum miR-155 levels were observed in all three models. For example, increased serum miR-155 levels in alcohol-fed mice after four weeks of alcohol feeding were observed (FIG. 9). However, there was no serum miR-155 increase in alcohol-fed TLR4 KO or p47^{phox} KO mice that were protected from alcohol-induced liver inflammation.

These data demonstrate the novel finding that serum/plasma miR-122 up-regulation correlates with serum ALT; thus, miR-122 is useful as a biomarker in acute and chronic liver injury. This invention also provides, for the first time, that serum miR-155 is increased in liver disease with inflammation.

Thus, in view of the above results, serum miR-155 as an inflammation marker in other models of liver disease such as NASH model will also be evaluated.

### Example 7

### Serum microRNAs as Diagnostic Biomarkers

The instant invention provides that serum or plasma microRNA (miRs) can be exploited as diagnostic biomarkers for various diseases because of their increased stability in various body fluids and also high assay sensitivity. This invention analyzed serum and plasma miRs as potential biomarkers for the detection of liver injury caused by various stimuli such as alcohol, NASH, acetaminophen (APAP) or carbon tetrachloride (CCL4).

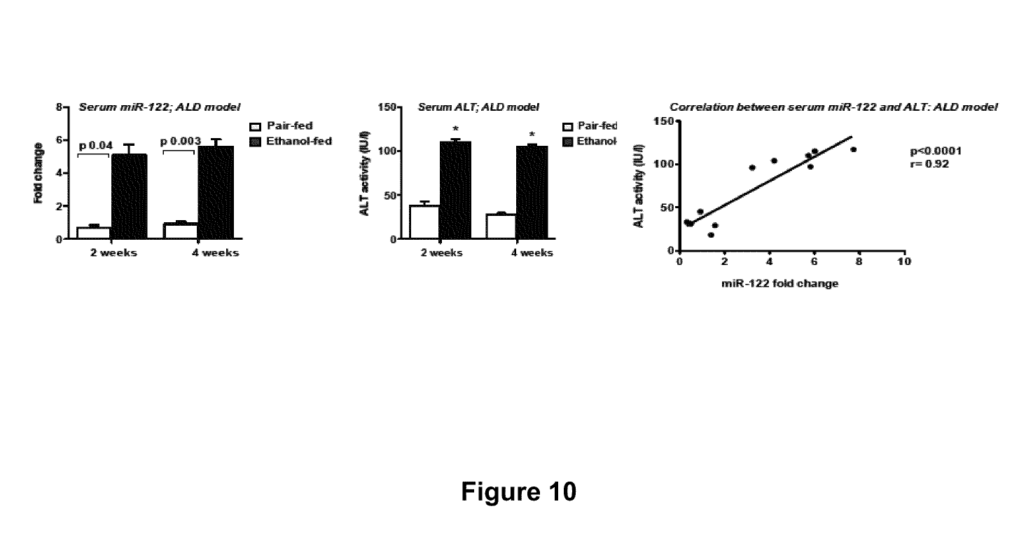

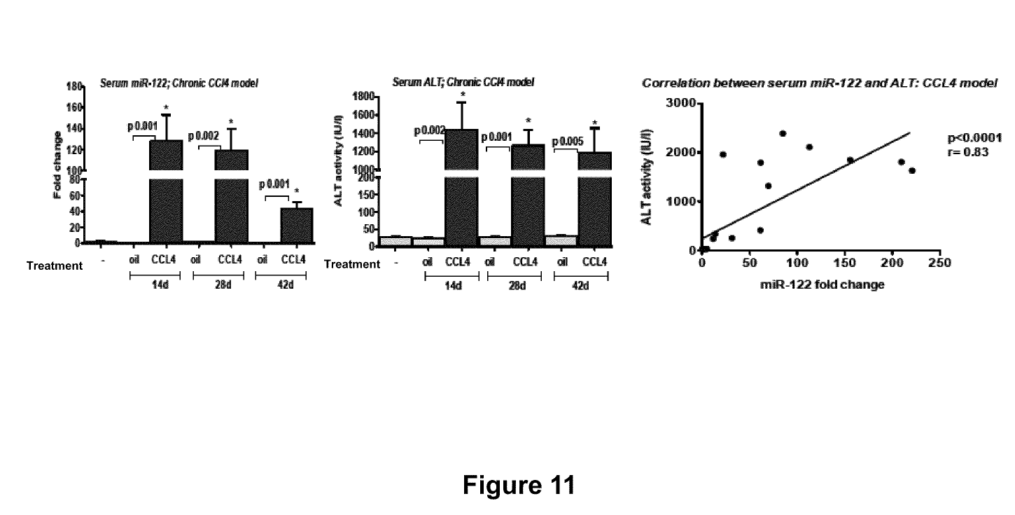

Increased serum miR-122 was observed after 2 or 4 weeks of alcohol feeding in mice (FIG. 10 left). A significant increase in ALT was also observed at these times (FIG. 10 middle), more important a significant correlation was found between serum miR-122 and ALT levels (FIG. 10 right). Thus, serum miR-122 could be exploited as an alternative biomarker for the liver damage. Next, it was determined if release of miR-122 to the serum is limited only to alcohol insult/use or whether it can be used as a universal marker for any kind of liver injury. To test this notion, a CCL4-induced liver injury mice model was used. In this study, CCl4 or corn oil (control) was administered via i.p. for 6 weeks. Interestingly, induction of miR-122 in the serum after 2 weeks of CCL4 administration was observed; however, this was not observed in the corn oil control. Furthermore, miR-122 remained elevated after 4 weeks and then gradually decreased after 6 weeks, but still remained significantly up-regulated (FIG. 11 left). Similar kinetics were found for ALT levels (FIG. 11 middle) and the correlation study indicates that ALT and serum miR-122 levels are positively correlated throughout CCL4-induced liver injury (FIG. 11 right).

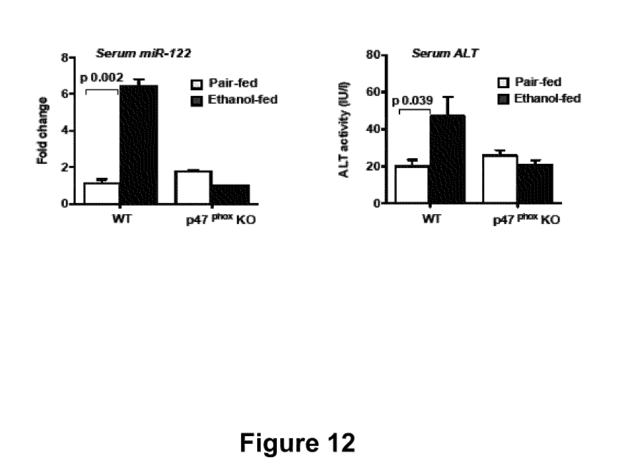

To prove that the serum miR-122 release takes place only after liver insult, NADPH oxidase-deficient (p47^{phox} KO) mice were examined. NADPH-p47^{phox} complex involves reactive oxygen species (ROS) and free radical generation. Interestingly, p47^{phox} KO mice were protected from alcoholic-induced liver injury, steatosis and inflammation; and thus, there was no increase in serum miR-122 level (FIG. 12 left). This observation was confirmed by measuring serum ALT levels of the corresponding mice, as no increase in ALT values was detected after 4 weeks of alcohol feeding (FIG. 12 right). This indicates that miR-122 release takes place in response to the liver injury and is correlated with ALT levels.

Thus, this invention demonstrates that miR-122 could be used as an alternative biomarker for the liver injury.

### Example 8

### TLR4 is Involved in the Alcohol Induced Expression of MiR-155

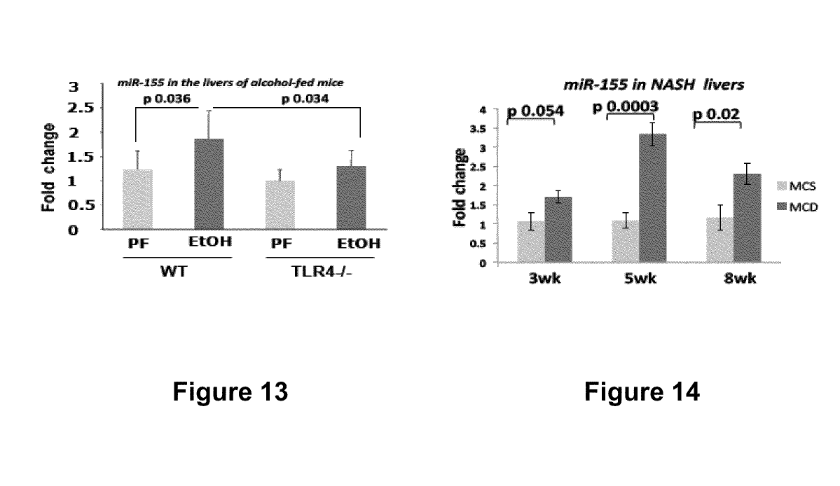

The mechanism by which alcohol induces miR-155 expression in the liver was also analyzed. TLR4 plays a crucial role in ALD and we have determined that TLR4 Knock-Out (KO) mice were protected from ALD, and also found the enhancement of miR-155 upon LPS treatment (TLR4 ligand) in Kupffer cells of alcohol-fed mice. It has now been determined that there is a significant reduction in miR-155 level in the livers of TLR4 KO mice after prolonged alcohol feeding. Thus, TLR4 is involved in the alcohol induced expression of miR-155 (FIG. 13).

### Example 9

### MiR-155 Functions in the Progression of Inflammation in MCD-Diet Fed Mice

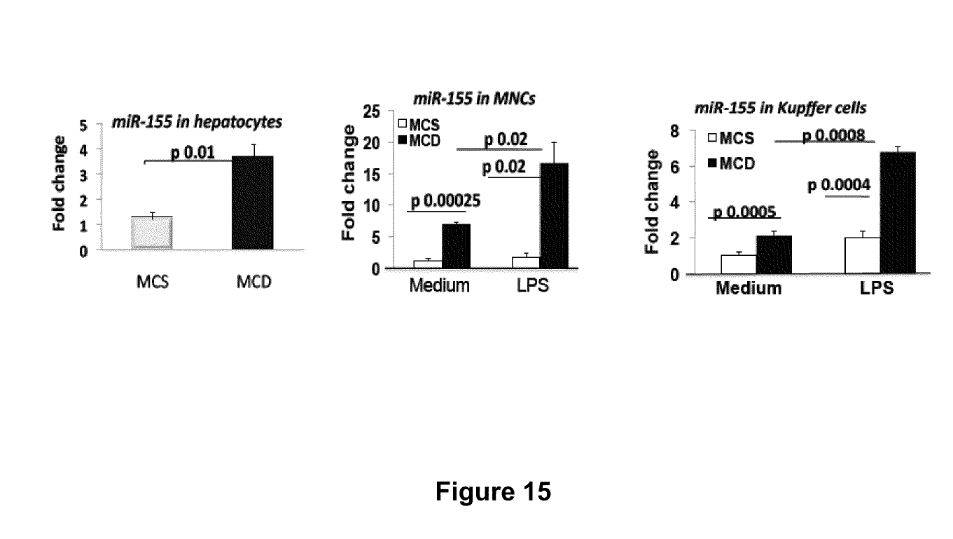

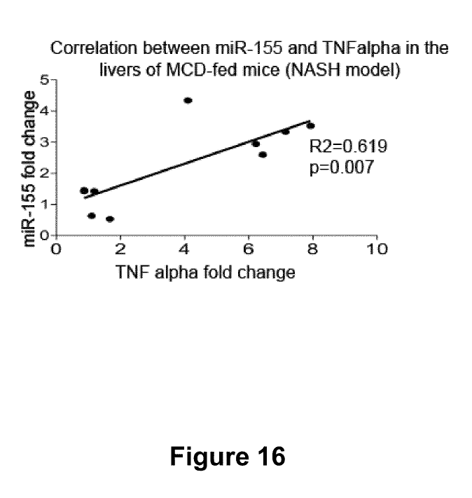

The role of miR-155 in non-alcoholic steatohepatitis (NASH), an increasingly common liver disease, was also investigated. The Methionine-choline-deficient (MCD) diet model was used to mimic human pathomechanisms of NASH with the Methionine-choline-sufficient (MCS) feeding control. After three weeks of feeding, increased levels of miR-155 were observed in the livers of MCD diet-fed mice; however, maximum induction was seen after 5 weeks and a gradual decrease was observed by 8 weeks (FIG. 14). The 5-week MCD diet induces a inflammation to a greater extent, whereas the 8-week MCD diet causes more fibrosis. These data indicate that miR-155 functions in the progression of liver inflammation. As different cell types play diverse roles in pathomechanisms of the disease, the level of miR-155 in different liver cells was assessed after 5-week of MCS/MCD diet. It was determined that there were elevated levels of miR-155 in hepatocytes (FIG. 15 left), in liver mononuclear cells (MNCs) (FIG. 15 middle) and Kupffer cells (FIG. 15 right) with and without 100 ng/ml LPS in vitro treatment for 6 h in MCD-fed mice. These data demonstrate the involvement of other cell types in inducing inflammation. In addition, a positive correlation between miR-155 and TNF alpha levels in the livers of MCD-fed mice was observed after 5 weeks of feeding (FIG. 16).

### Example 10

### Oral Delivery of miR-155 Antagonist in Animal Models of Inflammatory Liver Disease

To assess a therapeutic dosage regime for oral miR-155 antagonist therapy, an art-recognized animal or dietary model of inflammatory liver disease or disorder may be employed. For example, rats may be fed a choline-deficient, L-amino acid defined diet (Denda et al., Jpn. J. Cancer Res. 2002, 93: 125-132). Rodents on this diet show well-defined pathological changes of NASH. Alternatively, rats may be fed ethanol on a Lieber-DeCarli diet to approximate ALD.

To accomplish oral delivery of miR-155 antagonists to the liver of the animal model, micron-sized particles of β1,3-D-glucan may be employed (see Beier& Gebert, Am J Physiol 275, G130-7 (1998); Hong et al. J Immunol 173, 797-806 (2004), both of which are hereby incorporated by reference in their entirety). Hollow, porous micron-sized shells composed primarily of β1,3-D-glucan are prepared by treating baker's yeast with a series of alkaline, acid and solvent extractions to remove cytoplasmic components, as well as other cell wall polysaccharides (Soto, E. & Ostroff, G. R., NSTI Nanotech 2007 Technical Proceedings 2, 378-381 (2007), hereby incorporated by reference in its entirety). Such hollow glucan shells are about 2-4 microns in diameter. Layer by layer nanoparticle synthesis methods are then developed to load them with miR-155 antagonists, yielding β1,3-D-glucan-encapsulated particles (GeRPs).

Without being bound to any particular theory, it is thought that GeRPs undergo phagocytosis by resident macrophages and dendritic cells via the dectin-1 receptor and other beta glucan receptor-mediated pathways (Herre et al. Mol Immunol 40, 869-76 (2004); Willment et al., J Biol Chem 276, 43818-23 (2001), both of which are hereby incorporated by reference in their entirety), such that over time a significant proportion of total body macrophages (including liver Kupffer cells) contain ingested glucan particles. Upon phagocytosis by macrophages, GeRPs traffic to the endosomal compartment, where the acidic pH changes the layers' charge. This promotes nucleic acid release from the multi-layered nanoparticulate complex through the porous GeRP wall and endosomal membrane into the macrophage cytoplasm. Accordingly, to test GeRP formulations for effectiveness in miR-155 silencing, Kuppfer cells may be isolated and assayed for miR-155 and/or TNF expression levels relative to a suitable control.

1. A method for treating or preventing an inflammatory liver disease, comprising: identifying a subject having, or at risk of having, an inflammatory liver disease, and administering to the subject a miR-155 antagonist in an amount effective to decrease expression of miR-155 in the subject, wherein the miR-155 antagonist partially suppresses TNFα expression, thereby treating or preventing the disease.

2. The method of claim 1, wherein the inflammatory liver disease is selected from the group consisting of alcoholic liver disorder (ALD), non-alcoholic fatty liver disease (NAFLD) or non-alcoholic steatohepatitis (NASH).

3. The method of claim 2, wherein the inflammatory liver disease is ALD.

4. The method of claim 1, wherein said miR-155 antagonist is an anti-miR155 antisense oligonucleotide.

5. The method of claim 4, wherein the antisense oligonucleotide is an antagomir.

6. The method of claim 4, wherein the antisense oligonucleotide is an LNA oligonucleotide.

7. The method of claim 4, wherein the antisense oligonucletoide is an 2′O-methyl antisense RNA oligonucleotide.

8. The method of claim 1, wherein the miR-155 antagonist is an RNAi agent.

9. The method of claim 8, wherein the RNAi agent is a siRNA.

10. The method of claim 8, wherein the RNAi agent is a shRNA.

11. The method of claim 1, wherein said miR-155 antagonist is complementary to a sequence at least 80% identical to human mature miRNA-155.

12. The method of claim 1, wherein the miR-155 antagonist is complementary to a sequence at least 80% identical to pre-microRNA-155.

13. The method of claim 1, wherein the miR-155 antagonist is perfectly complementary to a human microRNA-155 seed sequence.

14. The method of claim 1, wherein the miR-155 is administered in a pharmaceutical composition comprising a yeast cell wall particle (YCWP).

15. A method for decreasing the stability of TNFα mRNA in a target cell, comprising administering a miR155 antagonist to the cell in an amount effective to decrease expression of miR-155 in the cell, thereby decreasing the stability of TNFα mRNA in the cell.

16. The method of claim 15, wherein the target cell is a Kupffer cell.

17. The method of claim 15, wherein the target cell is a macrophage.

18. The method of claim 15, wherein the antisense oligonucleotide is an antagomir.

19. The method of claim 18, wherein the antisense oligonucleotide is an LNA oligonucleotide.

20. The method of claim 18, wherein the antisense oligonucletoide is an 2′O-methyl antisense RNA oligonucleotide.

21. The method of claim 15, wherein the miR-155 antagonist is an RNAi agent.

22. The method of claim 21, wherein the RNAi agent is a siRNA.

23. The method of claim 21, wherein the RNAi agent is a shRNA.

24. The method of claim 15, wherein said miR-155 antagonist is complementary to a sequence at least 80% identical to human mature miRNA-155.

25. The method of claim 15, wherein the miR-155 antagonist is complementary to a sequence at least 80% identical to pre-microRNA-155.

26. The method of claim 15, wherein the miR-155 antagonist is perfectly complementary to a human microRNA-155 seed sequence.
